(19) United States
(12) Patent Application Publication
(10) Pub. No.: US 2008/0306045 A1
(43) Pub. Date: Dec. 11, 2008

(54) 1-Benzazepine-3-Sulfonylamino-2-Pyrroridones as Factor Xa Inhibitors

(76) Inventors: Laure Camus, Hertfordshire (GB); Reshma Chudasama, Hertfordshire (GB); Caroline Jane Day, Hertfordshire (GB); Deepali Deshmukh, Collegeville, PA (US); John Gerald Gleason, King of Prussia, PA (US); John David Harling, Hertfordshire (GB); Chao-Pin Lee, King of Prussia, PA (US); Paula Saklatvala, Hertfordshire (GB); Stefan Senger, Hertfordshire (GB); Sarah Vallance, Hertfordshire (GB); John Watson, Hertfordshire (GB); Nigel Stephen Watson, Hertfordshire (GB); Robert John Young, Hertfordshire (GB); Barbara Yuan, King of Prussia, PA (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/094,707

(22) PCT Filed: Nov. 22, 2006
(86) PCT No.: PCT/EP06/11218

§ 371 Date: May 22, 2008

(30) Foreign Application Priority Data
Nov. 24, 2005 (GB) 0523951.2
May 18, 2006 (GB) 0609900.6
Oct. 11, 2006 (GB) 0620155.2

(51) Int. Cl. A61K 31/55; C07D 409/14; C07D 403/10; C07D 403/14

(52) U.S. Cl. 514/213.01; 540/593

(57) ABSTRACT

The invention relates to chemical entities of formula (I):

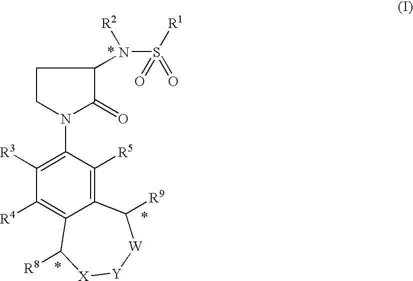

and/or pharmaceutically acceptable derivative(s) thereof. The invention also relates to processes for the preparation of compounds of formula (I), pharmaceutical compositions containing compounds of formula (I) and to the use of compounds of formula (I) in medicine, particularly in the amelioration of a clinical condition for which a Factor Xa inhibitor is indicated.

### FIELD OF THE INVENTION

The present invention relates to a novel class of chemical compounds, to processes for their preparation, to pharmaceutical compositions containing them and to their use in medicine, particularly use in the amelioration of a clinical condition for which a Factor Xa inhibitor is indicated.

### BACKGROUND OF THE INVENTION

Factor Xa is a member of the trypsin-like serine protease class of enzymes. It is a key enzyme in the coagulation cascade. A one-to-one binding of Factors Xa and Va with calcium ions and phospholipid converts prothrombin into thrombin. Thrombin plays a central role in the mechanism of blood coagulation by converting the soluble plasma protein, fibrinogen, into insoluble fibrin. The insoluble fibrin matrix is required for the stabilisation of the primary hemostatic plug. Many significant disease states are related to abnormal hemostasis. With respect to the coronary arterial vasculature, abnormal thrombus formation due to the rupture of an established atherosclerotic plaque is the major cause of acute myocardial infarction and unstable angina. Both treatment of an occlusive coronary thrombus by thrombolytic therapy and percutaneous transluminal coronary angioplasty (PTCA) are often accompanied by an acute thrombotic reclosure of the affected vessel which requires immediate resolution. With respect to the venous vasculature, a high percentage of patients undergoing major surgery in the lower extremities or the abdominal area suffer from thrombus formation in the venous vasculature which can result in reduced blood flow to the affected extremity and a pre-disposition to pulmonary embolism. Disseminated intravascular coagulopathy commonly occurs within both vascular systems during septic shock, certain viral infections and cancer and is characterised by the rapid consumption of coagulation factors and systemic coagulation which results in the formation of life-threatening thrombi occurring throughout the vasculature leading to widespread organ failure. Beyond its direct role in the formation of fibrin rich blood clots, thrombin has been reported to have profound bioregulatory effects on a number of cellular components within the vasculature and blood, (Shuman, M. A., Ann. NY Acad. Sci., 405: 349 (1986)).

A Factor Xa inhibitor may be useful in the treatment of acute vascular diseases such as acute coronary syndromes (for example primary and secondary prevention of myocardial infarction and unstable angina and treatment of prothrombotic sequalae associated with myocardial infarction or heart failure), thromboembolism, acute vessel closure associated with thrombolytic therapy and percutaneous transluminal coronary angioplasty, transient ischemic attacks, pulmonary embolism, deep vein thrombosis, peripheral arterial occlusion, prevention of vessel luminal narrowing (restenosis), and the prevention of thromboembolic events associated with atrial fibrillation, e.g. stroke. Factor Xa inhibitors may also be useful in preventing thrombosis and complications in patients genetically predisposed to arterial thrombosis or venous thrombosis and patients that have a disease-associated predisposition to thrombosis (e.g. type 2 diabetics). Thrombin has been reported to contribute to lung fibroblast proliferation, thus, Factor Xa inhibitors could be useful for the treatment of some pulmonary fibrotic diseases. Factor Xa inhibitors could also be useful in the treatment of tumour metastasis, by suppressing coagulation and thus preventing fibrin deposition and its concommittant facilitation of metastasis. A Factor Xa inhibitor may also have utility as an anti-inflammatory agent through its inhibition of FXa mediated activation of protease-activated receptors (PAR 1-4). A Factor Xa inhibitor may also have utility as an anti-atherosclerotic agent through the suppression of platelet-activation. Thrombin can induce neurite retraction and thus Factor Xa inhibitors may have potential in neurogenerative diseases such as Parkinson's and Alzheimer's disease. Factor Xa inhibitors may also have utility as anticoagulant agents in connection with the preparation, storage, fractionation or use of whole blood. They have also been reported for use in conjunction with thrombolytic agents, thus permitting the use of a lower dose of thrombolytic agent.

### DESCRIPTION OF THE INVENTION

The present invention provides at least one chemical entity chosen from compounds of formula (I):

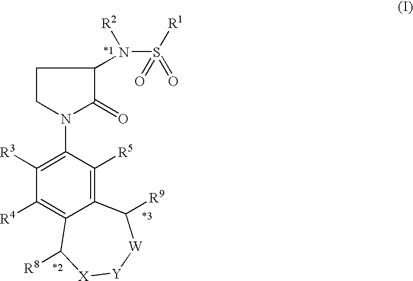

wherein:
R¹ represents a group selected from:

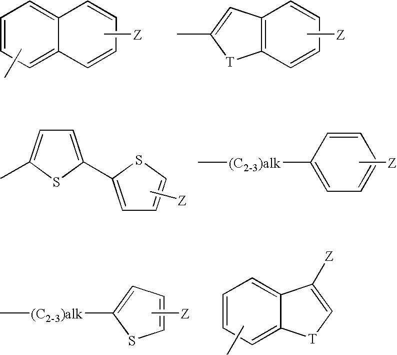

each ring of which optionally contains a further heteroatom N,
Z represents an optional substituent halogen,
alk represents alkylene or alkenylene, and
T represents S, O or NH;
R² represents hydrogen, —C₁₋₆alkyl, —C₁₋₃alkylCONR^{a}R^{b}, —C₁₋₃alkylCO₂C₁₋₄alkyl, —CO₂C₁₋₄alkyl, —C₁₋₃alkylOH, —C₁₋₃alkylOC₁₋₃alkyl, or —C₁₋₃alkylCO₂H;
R^{a} and R^{b} independently represent hydrogen, —C₁₋₆alkyl, or together with the N atom to which they are bonded form a 5-, 6- or 7-membered non-aromatic heterocyclic ring optionally containing an additional heteroatom selected from O, N and S, optionally substituted by C₁₋₄ alkyl, and optionally the S heteroatom is substituted by 0, i.e. represents S(O)ₙ;
n represents 0-2;
One of W, X and Y represents —N(R⁶)— and the others represent —CH(R⁷)—.
R³, R⁴ and R⁵ independently represent hydrogen, C₁₋₄alkyl or halogen.
R⁶ represents hydrogen or C₁₋₄alkyl.
R⁷, R⁸ and R⁹ independently represent hydrogen or C₁₋₄alkyl.
and pharmaceutically acceptable derivative(s) thereof.

The compounds of formula (I) contain chiral (asymmetric) centres (as indicated by the symbol *¹, *² and *³). The individual stereoisomers (enantiomers and diastereoisomers) and mixtures of these are within the scope of the present invention. In one aspect of the invention, the stereochemistry is (S) at the 3-position on the 2-oxopyrrolidine ring (as indicated by the symbol *¹ in formula (I)). It will be appreciated that compounds of formula (I) may also contain additional chiral centres. For example, the compounds of formula (I) also contain chiral (asymmetric) centres when X, Y and/or W represents —CH(R⁷)— and R⁷ is other than hydrogen.

Further aspects of the invention are:
A pharmaceutical composition comprising at least one chemical entity chosen from compounds of formula (I) and pharmaceutically acceptable derivative(s) thereof together with a pharmaceutical carrier and/or excipient.
At least one chemical entity chosen from compounds of formula (I) and pharmaceutically acceptable derivative(s) thereof for use in therapy.
Use of at least one chemical entity chosen from compounds of formula (I) and pharmaceutically acceptable derivative(s) thereof for the manufacture of a medicament for the treatment of a patient suffering from a condition susceptible to amelioration by a Factor Xa inhibitor.
At least one chemical entity chosen from compounds of formula (I) and pharmaceutically acceptable derivative(s) thereof for use in the treatment of a patient suffering from a condition susceptible to amelioration by a Factor Xa inhibitor.
A method of treating a patient suffering from a condition susceptible to amelioration by a Factor Xa inhibitor comprising administering a therapeutically effective amount of at least one chemical entity chosen from compounds of formula (I) and pharmaceutically acceptable derivative(s) thereof.

In one aspect of the invention, R¹ represents a group selected from:

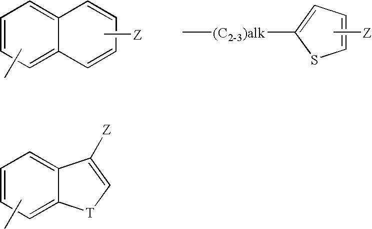

each ring of which optionally contains a further heteroatom N,
Z represents an optional substituent halogen,
alk represents alkylene or alkenylene, and
T represents S, O or NH.

In another aspect, R¹ represents a group selected from:

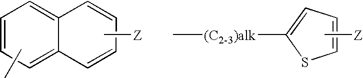

each ring of which optionally contains a further heteroatom N,
Z represents an optional substituent halogen, and
alk represents alkylene or alkenylene.

In another aspect, R¹ represents a group selected from:

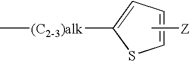

Z represents an optional substituent halogen, and
alk represents alkylene or alkenylene.

Alternatively, R¹ represents a group selected from:

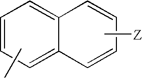

Z represents an optional substituent halogen.

Alternatively, R¹ represents a group selected from:

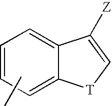

Z represents an optional substituent halogen,
alk represents alkylene or alkenylene, and
T represents NH.

Alternatively, R¹ represents a group selected from:

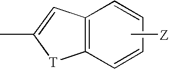

Z represents an optional substituent halogen, and
T represents S.

Alternatively, R¹ represents a group selected from:

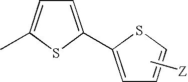

Z represents an optional substituent halogen.

R¹ represents a group selected from:

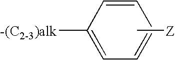

Z represents an optional substituent halogen, and
alk represents alkylene or alkenylene.

In another aspect of the invention, R¹ represents a group selected from:

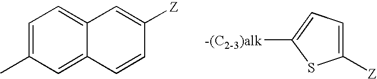

Z represents an optional substituent halogen.

In one aspect of the invention, Z represents fluorine or chlorine. In another aspect of the invention, Z represents chlorine.

In one aspect of the invention, T represents S or N. In another aspect of the invention, T represents S. In another aspect of the invention, T represents N.

In one aspect of the invention, R² represents hydrogen, —C₁₋₆alkyl, —C₁₋₃alkylCO₂C₁₋₄alkyl, —C₁₋₃ alkylOH or —C₁₋₃alkylCO₂H. In another aspect of the invention, R² represents hydrogen, —C₁₋₆ alkyl, —C₁₋₃alkylCO₂C₁₋₄alkyl, or —C₁₋₃alkylCO₂H. In another aspect of the invention R² represents —C₁₋₃alkylCO₂C₁₋₄alkyl, —C₁₋₃alkylOH or —C₁₋₃alkylCO₂H. In another aspect of the invention R² represents —CH₂CO₂C₁₋₁₄alkyl, —C₂H₂C₂H₂OH or —CH₂CO₂H. In another aspect of the invention R² represents —CH₂CO₂C₂H₅, or —CH₂CO₂H. In another aspect of the invention R² represents —CH₂CO₂CH(CH₃)₂ or —CH(CH₃)CO₂C₂H₅. In another aspect of the invention R² represents —CH₂CO₂C₂H₅. In another aspect of the invention, R² represents hydrogen or —C₁₋₆ alkyl. In another aspect of the invention, R² represents hydrogen or methyl. In another aspect of the invention, R² represents hydrogen.

In one aspect of the invention, R^{a} and R^{b} independently represent hydrogen or —C₁₋₆alkyl.

In one aspect of the invention, n represents 0-2.

In one aspect of the invention, R³ represents hydrogen, methyl or fluorine. In another aspect of the invention, R³ represents hydrogen.

In one aspect of the invention, R⁴ represents hydrogen, methyl or fluorine. In another aspect of the invention, R⁴ represents hydrogen.

In one aspect of the invention, R⁵ represents hydrogen, methyl or fluorine. In another aspect of the invention, R⁵ represents hydrogen.

In one aspect of the invention, R⁶ represents hydrogen or methyl. In another aspect of the invention, R⁶ represents hydrogen.

In one aspect of the invention, R⁷ represents hydrogen or methyl. In another aspect of the invention, R⁷ represents hydrogen.

In one aspect of the invention, R⁸ represents hydrogen or methyl. In another aspect of the invention, R⁸ represents hydrogen.

In one aspect of the invention, R⁹ represents hydrogen or methyl. In another aspect of the invention, R⁹ represents hydrogen.

In one aspect of the invention, one of W, X and Y represents —NH— and the others represent —CH₂—. In another aspect of the invention, X represents —CH(R⁷)—, one of W and Y represents —N(R⁶)— and the other represents —CH(R⁷)—. In another aspect of the invention, Y represents —CH(R⁷)—, one of X and W represents —N(R⁶)— and the other represents —CH(R⁷)—. In another aspect of the invention, W represents —CH(R⁷)—, one of X and Y represents —N(R⁶)— and the other represents —CH(R⁷)—. In another aspect of the invention, X represents —N(R⁶)— and Y and W represent —CH₂—. In another aspect of the invention, Y represents —N(R⁶)— and X and W represent —CH₂—. In another aspect of the invention, W represents —N(R⁶)— and X and Y represent —CH₂—.

It is to be understood that the present invention covers all combinations of the various aspects of the invention described herein above.

As used herein, the term “alkyl” means both straight and branched chain saturated hydrocarbon groups. Examples of alkyl groups include methyl (—CH₃), ethyl (—C₂H₅), propyl (—C₃H₇) and butyl (—C₄H₉).

As used herein, the term “alkylene” means both straight and branched chain saturated hydrocarbon linker groups. Examples of alkylene groups include methylene (—CH₂—), ethylene (—CH₂CH₂—) and propylene (—CH₂CH₂CH₂—).

As used herein, the term “alkenylene” means both straight and branched chain unsaturated hydrocarbon linker groups, wherein the unsaturation is present only as double bonds. Examples of alkenylene groups includes ethenylene (—CH═CH—) and propenylene (—CH₂—CH═CH—).

As used herein, the term “non-aromatic heterocyclic ring” means optionally substituted rings containing one or more heteroatoms selected from: nitrogen, sulphur and oxygen atoms. Examples of 5-membered groups include pyrrolidinyl. Examples of 6-membered rings include piperidinyl and morpholinyl. Examples of 7-membered rings include hexamethyleneiminyl.

As used herein, the term “halogen” means an atom selected from fluorine, chlorine, bromine and iodine.

As used herein, the term “pharmaceutically acceptable” means a compound which is suitable for pharmaceutical use.

As used herein, the term “pharmaceutically acceptable derivative”, means any pharmaceutically acceptable salt, solvate, or prodrug e.g. ester or carbamate, or salt or solvate of such a prodrug, of a compound of formula (I), which upon administration to the recipient is capable of providing (directly or indirectly) a compound of formula (I), or an active metabolite or residue thereof. Exemplary pharmaceutically acceptable derivatives are salts, solvates, esters and carbamates. More exemplary pharmaceutically acceptable derivatives are salts, solvates and esters. Even more exemplary pharmaceutically acceptable derivatives are salts and solvates.

Suitable salts according to the invention may include those formed with both organic and inorganic acids and bases. Pharmaceutically acceptable acid addition salts include those formed from mineral acids such as: hydrochloric, hydrobromic, benzoic, sulphuric, phosphoric, acid; and organic acids such as: citric, tartaric, lactic, pyruvic, acetic, trifluoroacetic, succinic, oxalic, formic, fumaric, maleic, oxalacetic, methanesulphonic, ethanesulphonic, p-toluenesulphonic, benzenesulphonic and isethionic acids. Exemplary pharmaceutically acceptable salts include hydrochloric, hydrobromic, benzoic and succinic acids. Exemplary pharmaceutically acceptable salts include those formed from hydrochloric and formic acids. Thus, in one aspect of the invention, pharmaceutically acceptable salts are hydrochloric acid salts. In another aspect of the invention, pharmaceutically acceptable salts are succinic acid salts (succinate e.g. hemisuccinate).

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compound of formula (I) are within the scope of the invention.

Salts and solvates of compounds of formula (I) which are suitable for use in medicine may be those wherein the counterion or associated solvent is pharmaceutically acceptable. However, salts and solvates having non-pharmaceutically acceptable counterions or associated solvents are within the scope of the present invention, for example, for use as intermediates in the preparation of other compounds of formula (I) and their pharmaceutically acceptable salts and solvates.

It will also be appreciated that compounds of the invention which exist as polymorphs, and mixtures thereof, are within the scope of the present invention.

As used herein, the term “prodrug” means a compound which is converted within the body, e.g. by hydrolysis in the blood, into its active form that has medical effects. Pharmaceutically acceptable prodrugs are described in T. Higuchi and V. Stella, Prodrugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and in D. Fleisher, S. Ramon and H. Barbra “Improved oral drug delivery: solubility limitations overcome by the use of prodrugs”, Advanced Drug Delivery Reviews (1996) 19(2) 115-130, each of which are incorporated herein by reference.

Prodrugs are any covalently bonded carriers that release a compound of structure (I) in vivo when such prodrug is administered to a patient. Prodrugs are generally prepared by modifying functional groups in a way such that the modification is cleaved in vivo yielding the parent compound. Prodrugs may include, for example, compounds of this invention wherein amine groups are bonded to any group that, when administered to a patient, cleaves to form the amine groups.

Esters may be active in their own right and/or be hydrolysable under in vivo conditions in the human body. Suitable pharmaceutically acceptable in vivo hydrolysable ester groups include those which break down readily in the human body to leave the parent acid or its salt. An ester may be formed at a carboxylic acid (—COOH) group, by methods well known in the art involving reaction with the corresponding alcohol. For example, esters may be C₁₋₆alkyl esters, e.g. methyl esters, ethyl esters, and the like.

As used herein, the term “compounds of the invention” means the compounds according to Formula I and the pharmaceutically acceptable derivative(s) thereof. The term “a compound of the invention” means any one of the compounds of the invention as defined above.

As used herein the term “at least one chemical entity” means at least one chemical substance chosen from the group of compounds consisting of compounds of Formula I and pharmaceutically acceptable derivative(s) thereof.

In one aspect, chemical entities useful in the present invention may be at least one chemical entity selected from the list:
(E)-2-(5-Chloro-2-thienyl)-N-[(3S)-2-oxo-1-(2,3,4,5-tetrahydro-1H-2-benzazepin-7-yl)-3-pyrrolidinyl]ethenesulfonamide;
6-Chloro-N-[(3S)-2-oxo-1-(2,3,4,5-tetrahydro-1H-2-benzazepin-7-yl)-3-pyrrolidinyl]-2-naphthalenesulfonamide;
3-Chloro-N-[(3S)-2-oxo-1-(2,3,4,5-tetrahydro-1H-2-benzazepin-7-yl)-3-pyrrolidinyl]-1H-indole-6-sulfonamide;
6-Chloro-N-[(3S)-1-(2-methyl-2,3,4,5-tetrahydro-1H-2-benzazepin-7-yl)-2-oxo-3-pyrrolidinyl]-2-naphthalenesulfonamide;
(E)-2-(5-Chloro-2-thienyl)-N-[(3S)-2-oxo-1-(2,3,4,5-tetrahydro-1H-3-benzazepin-7-yl)-3-pyrrolidinyl]ethenesulfonamide;
6-Chloro-N-[(3S)-2-oxo-1-(2,3,4,5-tetrahydro-1H-3-benzazepin-7-yl)-3-pyrrolidinyl]-2-naphthalenesulfonamide;
3-Chloro-N-[(3S)-2-oxo-1-(2,3,4,5-tetrahydro-1H-3-benzazepin-7-yl)-3-pyrrolidinyl]-1H-indole-6-sulfonamide;
(E)-2-(5-Chloro-2-thienyl)-N-[(3S)-2-oxo-1-(2,3,4,5-tetrahydro-1H-2-benzazepin-8-yl)-3-pyrrolidinyl]ethenesulfonamide;
6-Chloro-N-[(3S)-2-oxo-1-(2,3,4,5-tetrahydro-1H-2-benzazepin-8-yl)-3-pyrrolidinyl]-2-naphthalenesulfonamide;
(E)-2-(4-Chlorophenyl)-N-[(3S)-2-oxo-1-(2,3,4,5-tetrahydro-1H-2-benzazepin-8-yl)-3-pyrrolidinyl]ethenesulfonamide;
5′-Chloro-N-[(3S)-2-oxo-1-(2,3,4,5-tetrahydro-1H-2-benzazepin-8-yl)-3-pyrrolidinyl]-2,2′-bithiophene-5-sulfonamide;
5-Chloro-N-[(3S)-2-oxo-1-(2,3,4,5-tetrahydro-1H-2-benzazepin-8-yl)-3-pyrrolidinyl]-1-benzothiophene-2-sulfonamide;
(E)-2-(5-Chloro-2-thienyl)-N-methyl-N-[(3S)-2-oxo-1-(2,3,4,5-tetrahydro-1H-2-benzazepin-7-yl)-3-pyrrolidinyl]ethenesulfonamide;
Ethyl N-{[(E)-2-(5-chloro-2-thienyl)ethenyl]sulfonyl}-N-[(3S)-2-oxo-1-(2,3,4,5-tetrahydro-1H-2-benzazepin-7-yl)-3-pyrrolidinyl]glycinate;
1,1-Dimethylethyl N-{[(E)-2-(5-chloro-2-thienyl)ethenyl]sulfonyl}-N-[(3S)-2-oxo-1-(2,3,4,5-tetrahydro-1H-2-benzazepin-7-yl)-3-pyrrolidinyl]glycinate;
N-{[(E)-2-(5-Chloro-2-thienyl)ethenyl]sulfonyl}-N-[(3S)-2-oxo-1-(2,3,4,5-tetrahydro-1H-2-benzazepin-7-yl)-3-pyrrolidinyl]glycine;
(E)-2-(5-Chloro-2-thienyl)-N-(2-hydroxyethyl)-N-[(3S)-2-oxo-1-(2,3,4,5-tetrahydro-1H-2-benzazepin-7-yl)-3-pyrrolidinyl]ethenesulfonamide;
1-Methylethyl N-{[(E)-2-(5-chloro-2-thienyl)ethenyl]sulfonyl}-N-[(3S)-2-oxo-1-(2,3,4,5-tetrahydro-1H-2-benzazepin-7-yl)-3-pyrrolidinyl]glycinate;
Ethyl N-{[(E)-2-(5-chloro-2-thienyl)ethenyl]sulfonyl}-N-[(3S)-2-oxo-1-(2,3,4,5-tetrahydro-1H-2-benzazepin-7-yl)-3-pyrrolidinyl]alaninate;
(E)-2-(5-Chloro-2-thienyl)-N-[(3S)-1-(6-fluoro-2,3,4,5-tetrahydro-1H-2-benzazepin-7-yl)-2-oxo-3-pyrrolidinyl]ethenesulfonamide;
6-Chloro-N-[(3S)-1-(6-fluoro-2,3,4,5-tetrahydro-1H-2-benzazepin-7-yl)-2-oxo-3-pyrrolidinyl]-2-naphthalenesulfonamide;
   and pharmaceutically acceptable derivative(s) thereof.

In one aspect of the invention there is provided substantially crystalline (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride form 1. In another aspect of the invention there is provided substantially crystalline (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride form 2. In another aspect of the invention there is provided substantially crystalline (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride hydrate. In another aspect of the invention there is provided substantially crystalline (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hemisuccinate.

Thermal analysis on samples of substantially crystalline (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride forms 1 and 2 and substantially crystalline (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hemisuccinate were carried out. Thus, there is provided substantially crystalline (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride (form 1 or form 2) having a melting point onset measured by DSC (±0.5° C.) of: 250° C. or greater, for example in the range 260-280° C. Thus, there is also provided substantially crystalline (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hemisuccinate having a melting point onset measured by DSC (±0.5° C.) of: 190° C. or greater, for example in the range 190-230° C., or in the range 210-230° C.

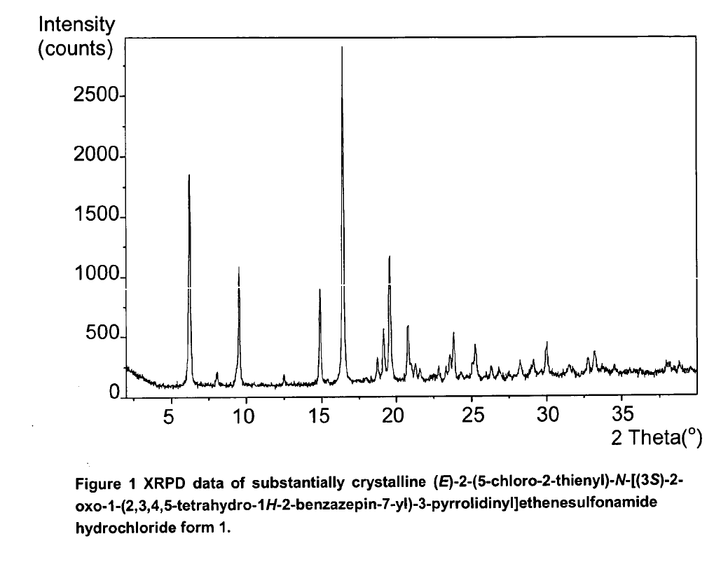

Samples of substantially crystalline (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride form 1, (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride form 2, (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride hydrate and substantially crystalline (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hemisuccinate, prepared as described hereinafter, gave the X-ray powder diffraction patterns of FIGS. 1-5. The X-ray diffraction pattern is unique to the crystalline form. The substantially crystalline forms exhibit diffraction patterns with a unique set of diffraction peaks which can be expressed in 2 theta angles (°) and/or d-spacings (Å).

2 Theta diffraction angles and corresponding d-spacing values account for positions of various peaks in the X-ray diffraction pattern. D-spacing values are calculated with observed 2 theta angles and copper Kα1 wavelength using the Bragg equation. Slight variations in observed 2 theta angles and d-spacings are expected based on the specific diffractometer employed and the analyst's sample preparation technique.

The substantially crystalline forms of (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride, (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride hydrate and (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hemisuccinate can be identified by the presence of a characteristic 2 theta angle peak or d-spacing, or by multiple 2 theta angles or d-spacings which are reasonably characteristic of the particular crystalline forms. To identify substantially crystalline (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride (form 1), these peaks occur at the following positions, expressed in 2 theta angles (±0.1 degrees): 8.0, 9.5, 19.6, 23.6 degrees or the following d-spacings (±0.1 Å): 11.0, 9.3, 4.5, 3.8. To identify substantially crystalline (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride (form 2), these peaks occur at the following positions, expressed in 2 theta angles (±0.1 degrees): 7.4, 8.9, 12.1, 14.7 degrees or the following d-spacings (±0.1 Å): 12.0, 9.9, 7.3, 6.0. To identify substantially crystalline (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride hydrate, these peaks occur at the following positions, expressed in 2 theta angles (±0.1 degrees): 5.7, 7.0, 8.5, 13.0, 23.9 degrees or the following d-spacings (±0.1 Å): 15.4, 12.7, 10.4, 6.8, 3.7. To identify substantially crystalline (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hemisuccinate, these peaks occur at the following positions, expressed in 2 theta angles (±0.1 degrees): 4.5, 8.7, 9.1, 10.4, 11.3, 13.3, 14.6, 17.1, 18.1, 18.7, 20.0, 21.1, 22.5, 22.9, 24.5, 25.5, 26.4, 27.2, 28.0, 29.6 or the following d-spacings (±0.1 Å): 19.7, 10.2, 9.8, 8.5, 7.8, 6.6, 6.1, 5.2, 4.9, 4.8, 4.4, 4.2, 4.0, 3.9, 3.6, 3.5, 3.4, 3.3, 3.2, 3.0

In one embodiment at least one of the foregoing 2 theta angles or d-spacings are employed to identify substantially crystalline (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride form 1, substantially crystalline (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride form 2, substantially crystalline (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride hydrate or (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hemisuccinate. In other embodiments at least 2, 3, 4 or 5 (where applicable) of the foregoing 2 theta angles or d-spacings are employed to identify substantially crystalline (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride form 1, substantially crystalline (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride form 2, substantially crystalline (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride hydrate or (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hemisuccinate

Some margin of error is present in each of the 2 theta angle assignments and d-spacings reported above. The error in determining d-spacings decreases with increasing diffraction scan angle or decreasing d-spacing. The margin of error in the foregoing 2 theta angles is approximately ±0.1 degrees, preferably ±0.05 degrees, for each of the foregoing peak assignments. The margin of error in d-spacing values is approximately ±0.1 Angstroms, preferably ±0.05 Angstroms.

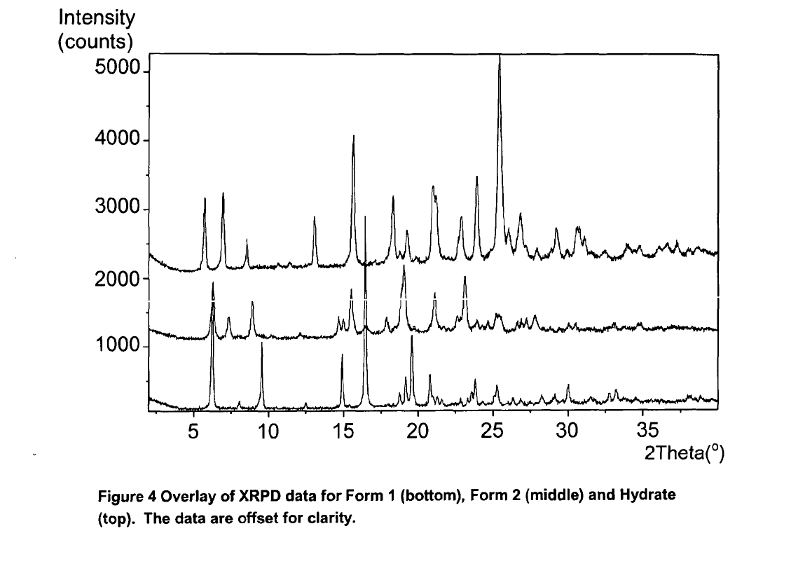

Since some margin of error is possible in the assignment of 2 theta angles and d-spacings, the preferred method of comparing X-ray powder diffraction patterns in order to identify a particular crystalline form is to overlay the X-ray powder diffraction pattern of the unknown form over the X-ray powder diffraction pattern of a known form. For example, one skilled in the art can overlay an X-ray powder diffraction pattern of an unidentified form of (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride or (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride hydrate, obtained using the methods described herein, see FIG. 4 for example, and readily determine whether the X-ray diffraction pattern of the unidentified form is substantially the same as the X-ray powder diffraction pattern of substantially crystalline (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride or (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride hydrate. If the X-ray powder diffraction pattern is substantially the same as that shown in any of FIGS. 1-3, the previously form can be readily and accurately identified.

As used herein, the term “substantially crystalline” means that the compound, e.g. (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride and/or (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hemisuccinate is substantially free of the amorphous form. By “substantially free” is meant containing less than 50% of the amorphous form, in one aspect less than 20% of the amorphous form, in another aspect less than 10% of the amorphous form, in another aspect less than 5% of the amorphous form, in another aspect less than 2% of the amorphous form, in another aspect less than 1% of the amorphous form.

The present invention provides a method for the preparation of substantially crystalline (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride form 1 from 1,1-dimethylethyl 7-[(3S)-3-({[(E)-2-(5-chloro-2-thienyl)ethenyl]sulfonyl}amino)-2-oxo-1-pyrrolidinyl]-1,3,4,5-tetrahydro-2H-2-benzazepine-2-carboxylate by reaction with HCl in a suitable solvent e.g. dioxane, followed by crystallisation of the derived solid by refluxing in an organic solvent e.g. EtOH and water, followed by cooling and isolation of the crystals by filtration. Optionally, further steps of suspension in an organic solvent e.g. EtOH followed by filtration may be carried out.

The present invention provides a method for the preparation of substantially crystalline (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride form 2 by dissolving (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide in an organic solvent e.g. EtOH, suitably at elevated temperature e.g. 40-50° C., followed by treatment with HCl in a suitable solvent e.g. diethyl ether, suitably at room temperature, and isolation of the crystals by filtration.

The present invention provides a method for the preparation of substantially crystalline (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride hydrate, from 1,1-dimethylethyl 7-[(3S)-3-({[(E)-2-(5-chloro-2-thienyl)ethenyl]sulfonyl}amino)-2-oxo-1-pyrrolidinyl]-1,3,4,5-tetrahydro-2H-2-benzazepine-2-carboxylate by reaction with HCl in a suitable solvent e.g. dioxane, followed by suspension of the derived solid in an organic solvent e.g. EtOH, and stirring for a suitable time (e.g. 1 h) suitably at room temperature, and isolation of the crystals by filtration.

The present invention provides a method for the preparation of substantially crystalline (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hemisuccinate from (E)-2-(5-Chloro-2-thienyl)-N-[(3S)-2-oxo-1-(2,3,4,5-tetrahydro-1H-2-benzazepin-7-yl)-3-pyrrolidinyl]ethenesulfonamide by reaction with succinic acid in a suitable solvent e.g. MeOH.

The present invention provides a method for the conversion of a mixture of substantially crystalline (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride form 2 and form 1 into substantially crystalline (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride form 1 by suspending the mixture in a suitable organic solvent (e.g. EtOH) and slurrying at a suitable temperature (e.g. room temperature to 50° C.) for a suitable time (e.g. 7 days).

The methods for the preparation of substantially crystalline material described herein constitute a further aspect of the present invention.

Compounds of the invention may show advantageous properties, they may be more efficacious, may show greater selectivity, may have fewer side effects, may have a longer duration of action, may be more bioavailable by the preferred route, or may have other more desirable properties than similar known compounds.

The compounds of formula (I) are Factor Xa inhibitors and as such are useful in the treatment of clinical conditions susceptible to amelioration by administration of a Factor Xa inhibitor. Such conditions may include acute vascular diseases such as acute coronary syndromes (for example primary and secondary prevention of myocardial infarction and unstable angina and treatment of prothrombotic sequalae associated with myocardial infarction or heart failure), thromboembolism, acute vessel closure associated with thrombolytic therapy and percutaneous transluminal coronary angioplasty (PTCA), transient ischemic attacks, pulmonary embolism, deep vein thrombosis, peripheral arterial occlusion, prevention of vessel luminal narrowing (restenosis), and the prevention of thromboembolic events associated with atrial fibrillation, e.g. stroke; in preventing thrombosis and complications in patients genetically predisposed to arterial thrombosis or venous thrombosis and patients that have a disease-associated predisposition to thrombosis (e.g. type 2 diabetics); the treatment of pulmonary fibrosis; the treatment of tumour metastasis; inflammation; atherosclerosis; neurogenerative disease such as Parkinson's and Alzheimer's diseases; Kasabach Merritt Syndrome; Haemolytic uremic syndrome; endothelial dysfunction; as anti-coagulants for extracorporeal blood in for example, dialysis, blood filtration, bypass, and blood product storage; and in the coating of invasive devices such as prostheses, artificial valves and catheters in reducing the risk of thrombus formation. Accordingly, one aspect of the present invention provides at least one chemical entity chosen from compounds of formula (I) and pharmaceutically acceptable derivative(s) thereof for use in medical therapy, for example, for use in the amelioration of a clinical condition in a mammal, including a human, for which a Factor Xa inhibitor is indicated.

In another aspect, the present invention provides the use of at least one chemical entity chosen from compounds of formula (I) and pharmaceutically acceptable derivative(s) thereof, for the manufacture of a medicament for the treatment and/or prophylaxis of a condition susceptible to amelioration by a Factor Xa inhibitor.

In another aspect, the invention provides a chemical entity chosen from compounds of formula (I) and pharmaceutically acceptable derivative(s) thereof for use in the treatment of a patient suffering from a condition susceptible to amelioration by a Factor Xa inhibitor.

In another aspect, the invention provides a method for the treatment and/or prophylaxis of a condition susceptible to amelioration by a Factor Xa inhibitor in a mammal, including a human, which method comprises administering to the subject an effective amount of at least one chemical entity chose from compounds of formula (I) and pharmaceutically acceptable derivative(s) thereof.

In one aspect of the invention, the condition susceptible to amelioration by a Factor Xa inhibitor is selected from treatment of acute vascular diseases such as acute coronary syndromes (for example primary and secondary prevention of myocardial infarction and unstable angina and treatment of prothrombotic sequalae associated with myocardial infarction or heart failure), thromboembolism, acute vessel closure associated with thrombolytic therapy and percutaneous transluminal coronary angioplasty, transient ischemic attacks, pulmonary embolism, deep vein thrombosis, peripheral arterial occlusion, prevention of vessel luminal narrowing (restenosis), and the prevention of thromboembolic events associated with atrial fibrillation, e.g. stroke.

In another aspect, the condition susceptible to amelioration by a Factor Xa inhibitor is selected from acute coronary syndromes (for example primary and secondary prevention of myocardial infarction and unstable angina and treatment of prothrombotic sequalae associated with myocardial infarction or heart failure), pulmonary embolism, deep vein thrombosis and the prevention of thromboembolic events associated with atrial fibrillation, e.g. stroke.

It will be appreciated that reference to treatment includes acute treatment or prophylaxis as well as the alleviation of established symptoms.

Within the context of the present invention, the terms describing the indications used herein are classified in the The Merck Manual of Diagnosis and Therapy, 17^{th} Edition and/or the International Classification of Diseases, 10th Edition (ICD-10). The various subtypes of the disorders mentioned herein are contemplated as part of the present invention.

While it is possible that, for use in therapy, an active ingredient may be administered as the raw chemical, the active ingredient may also be presented as a pharmaceutical formulation.

In a further aspect, the invention provides a pharmaceutical composition comprising at least one chemical entity chosen from compounds of formula (I) and pharmaceutically acceptable derivative(s) thereof in association with at least one pharmaceutically acceptable carrier and/or excipient. The carrier and/or excipient must be “acceptable” in the sense of being compatible with the other ingredients of the formulation and not deleterious to the recipient thereof.

In another aspect, the invention provides a pharmaceutical composition comprising at least one chemical entity chosen from compounds of formula (I) and pharmaceutically acceptable derivative(s) thereof in association with a pharmaceutically acceptable carrier and/or excipient for use in therapy, and for example in the treatment of human or animal subjects suffering from a condition susceptible to amelioration by a Factor Xa inhibitor.

There is further provided by the present invention a process of preparing a pharmaceutical composition, which process comprises mixing at least one chemical entity chosen from compounds of formula (I) and pharmaceutically acceptable derivative(s) thereof, together with at least one pharmaceutically acceptable carrier and/or excipient.

The compounds of the invention may be formulated for oral, buccal, parenteral, topical, rectal or transdermal administration or in a form suitable for administration by inhalation or insufflation (either through the mouth or the nose).

For oral administration, the pharmaceutical compositions may take the form of, for example, tablets or capsules prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycollate); or wetting agents (e.g. sodium lauryl sulphate). The tablets may be coated by methods well known in the art. Liquid preparations for oral administration may take the form of, for example, solutions, syrups or suspensions or they may be presented as a dry product for constitution with water or other suitable vehicles before use. Such liquid preparations may be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. almond oil, oily esters, ethyl alcohol or fractionated vegetable oils); and preservatives (e.g. methyl or propyl-p-hydroxybenzoates or sorbic acid). The preparations may also contain buffer salts, flavouring, colouring and sweetening agents as appropriate.

Preparations for oral administration may be suitably formulated to give controlled/extended release of the active compound.

For buccal administration the compositions may take the form of tablets or lozenges formulated in a conventional manner.

The compounds of the invention may be formulated for parenteral administration by injection, e.g. by bolus injection or continuous infusion. Formulations for injection may be presented in unit dosage form, e.g. in ampoules or in multi-dose containers, with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use.

The compounds of the invention may be formulated for topical administration by insufflation and inhalation. Examples of types of preparation for topical administration include sprays and aerosols for use in an inhaler or insufflator.

Powders for external application may be formed with the aid of any suitable powder base, for example, lactose, talc or starch. Spray compositions may be formulated as aqueous solutions or suspensions or as aerosols delivered from pressurised packs, such as metered dose inhalers, with the use of a suitable propellant.

The compounds of the invention may also be formulated in rectal compositions such as suppositories or retention enemas, e.g. containing conventional suppository bases such as cocoa butter or other glycerides.

In addition to the formulations described previously, the compounds may also be formulated as a depot preparation. Such long acting formulations may be administered by implantation (for example subcutaneously, transcutaneously or intramuscularly) or by intramuscular injection. Thus, for example, the compounds of the invention may be formulated with suitable polymeric or hydrophobic materials (for example as an emulsion in an acceptable oil) or ion exchange resins or as sparingly soluble derivatives, for example, as a sparingly soluble salt.

A proposed dose of the compounds of the invention for administration to a human (of approximately 70 kg body weight) is 0.1 mg to 1 g, such as 1 mg to 500 mg of the active ingredient per unit dose, expressed as the weight of free base. The unit dose may be administered, for example, 1 to 4 times per day. It will be appreciated that it may be necessary to make routine variations to the dosage depending on the age and weight of the patient as well as the severity of the condition to be treated. The dosage may also depend on the route of administration. The precise dose and route of administration will ultimately be at the discretion of the attendant physician or veterinarian.

The compounds of the invention may also be used in combination with other therapeutic agents. The invention thus provides, in a further aspect, a combination comprising at least one chemical entity chosen from compounds of formula (I) and pharmaceutically acceptable derivative(s) thereof together with one or more further therapeutic agent(s).

When at least one chemical entity chosen from compounds of formula (I) and pharmaceutically acceptable derivative(s) thereof is used in combination with a second therapeutic agent the dose of each compound may differ from that when the compound is used alone. Appropriate doses will be readily appreciated by those skilled in the art. It will be appreciated that the amount of a compound of the invention required for use in treatment will vary with the nature of the condition being treated and the age and the condition of the patient and will be ultimately at the discretion of the attendant physician or veterinarian. The compounds of the invention may be used in combination with other antithrombotic drugs (such as thrombin inhibitors, thromboxane receptor antagonists, prostacyclin mimetics, phosphodiesterase inhibitors, fibrinogen antagonists, thrombolytic drugs such as tissue plasminogen activator and streptokinase, non-steroidal anti-inflammatory drugs such as aspirin, and the like), anti-hypertensive agents (such as angiotensin-converting enzyme inhibitors, angiotensin-II receptor antagonists, ACE/NEP inhibitors, β-blockers, calcium channel blockers, PDE inhibitors, aldosterone blockers), anti-atherosclerotic/dyslipidaemic agents (such as HMG-CoA reductase inhibitors) and anti-arrhythmic agents.

The combinations referred to above may conveniently be presented for use in the form of a pharmaceutical formulation and thus pharmaceutical formulations comprising a combination as defined above together with at least one pharmaceutically acceptable carrier and/or excipient comprise a further aspect of the invention. The individual components of such combinations may be administered either sequentially or simultaneously in separate or combined pharmaceutical formulations by any convenient route.

When administration is sequential, either the Factor Xa inhibitor or the second therapeutic agent may be administered first. When administration is simultaneous, the combination may be administered either in the same or different pharmaceutical composition.

When combined in the same formulation it will be appreciated that the two compounds must be stable and compatible with each other and the other components of the formulation. When formulated separately they may be provided in any convenient formulation, conveniently in such manner as are known for such compounds in the art.

The chemical entities chosen from compounds of formula (I) and pharmaceutically acceptable derivative(s) thereof may be prepared by the processes described hereinafter, said processes constituting a further aspect of the invention. In the following description, the groups are as defined above for compounds of formula (I) unless otherwise stated.

According to a further aspect of the present invention, there is provided a process (A) for preparing compounds of formula (I) which comprises reacting compounds of formula (II) or an acid addition salt thereof with compounds of formula (III) where V is a suitable leaving group, such as a halide, e.g. chloride. The reaction is conveniently carried out in the presence of a base, e.g. pyridine, and in a suitable solvent, e.g. acetonitrile (MeCN), suitably at 0° C. to room temperature. In compounds of formula (II), P¹ represents an optional amine protecting group (on W, X or Y as appropriate). Where P¹ is a protecting group, e.g. t-butyloxycarbonyl (Boc), the reaction of compounds of formula (II) and compounds of formula (III) is followed by removal of the protecting group under standard conditions. For example, where P¹ represents Boc, removal of the protecting group may be effected under acidic conditions, for example, using a source of HCl, for example acetyl chloride in methanol (MeOH), HCl in dioxane or HCl in diethyl ether.

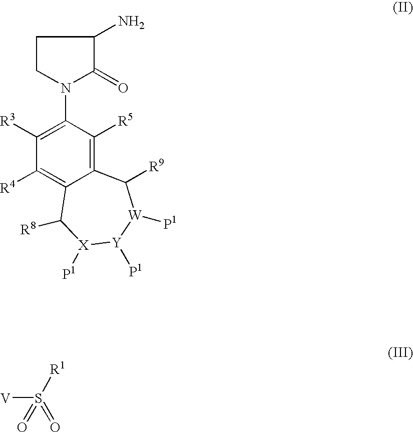

Where R¹ represents a nitrogen containing heterocycle, e.g. an indole, R¹ may be protected with a suitable amine protecting group which may be removed under standard conditions after the reaction between compounds of formula (II) with compounds of formula (III). For example, where the protecting group is tris(1-methylethyl)silyl this may be removed by fluoride deprotection, e.g. by treatment with tetraethylammonium fluoride in the presence of acetic acid and a suitable solvent, e.g. tetrahydrofuran (THF). For example, where the protecting group is Boc, this may be removed by acid deprotection, e.g. by treatment with HCl in MeOH.

Compounds of formula (III) are known compounds or may be prepared by methods known in the literature or processes known to those skilled in the art.

Compounds of formula (II) may be prepared from compounds of formula (IV)

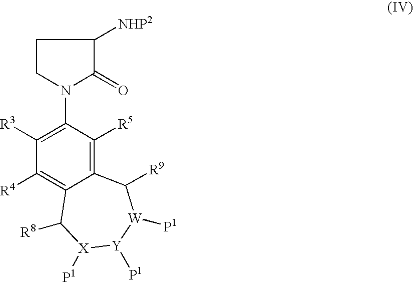

by removal of the protecting group P², under standard conditions. For example, where P² represents benzyloxycarbonyl (Cbz), removal of the protecting group may be effected by reaction with hydrogen in the presence of a metal catalyst, e.g. palladium/C or palladium hydroxide, in a suitable solvent e.g. ethanol (EtOH). For example, where P² represents Boc, removal of the protecting group may be effected under acidic conditions, using a source of HCl, for example acetyl chloride in MeOH.

Compounds of formula (IV) may be prepared from compounds of formula (V):

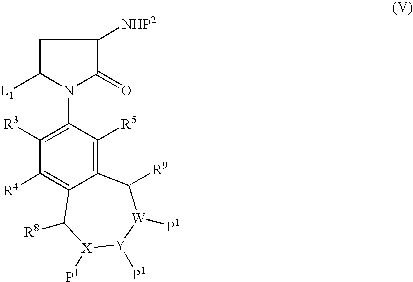

by cyclisation where L₁ represents a suitable group, e.g. hydroxyl, SMe. For example, when L₁ represents SMe by treatment with a compound capable of converting sulfur in the SMe moiety to a sulfonium salt, e.g. S⁺MeRX⁻, by reaction with RX (e.g. MeI), in a suitable solvent, e.g. MeCN, followed by ring closure. The ring closure may be performed with cesium carbonate (Cs₂CO₃) in a suitable solvent, e.g. MeCN, suitably at elevated temperature, such as 50-70° C. For example, where L₁ is a hydroxyl group, P¹ may be absent, the ring closure may be performed by treatment with a mixture of (i) aryl or alkyl phosphine, e.g. tri-n-butylphosphine, and (ii) a suitable azodicarboxylate derivative, e.g. di-tert-butyl azodicarboxylate, in a suitable solvent, e.g. THF, suitably at room temperature.

Compounds of formula (V) in which L₁ represents SMe may be prepared by reacting compounds of formula (VI) with compounds of formula (VII):

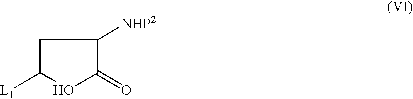

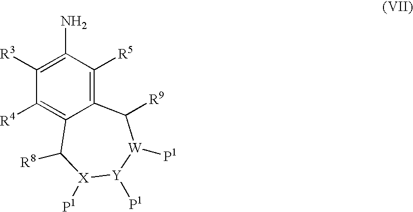

in the presence of a coupling agent, for example 2-(7-azabenzotriazole-1-yl)-1,1,3,3-tetramethyluronium hexafluorophosphate (HATU) and a base, e.g. N,N-diisopropylethylamine (DIPEA), in a suitable solvent, e.g. dichloromethane (DCM), suitably at 0° C. to room temperature.

Compounds of formula (VI) are known compounds or may be prepared by methods known in the literature or processes known to those skilled in the art.

Compounds of formula (VII) where YP¹ represents —NP¹— and XP¹ and WP¹ represent —CH(R⁷)— are known compounds or may be prepared by methods known in the literature or processes known to those skilled in the art.

It will be appreciated by persons skilled in the art that compounds of formula (VII) and protected derivatives thereof where YP¹ represents NP¹, XP¹ and WP¹ represent CHR⁷, or XP¹ represents NP¹, YP¹ represents CHR⁷ and WP¹ represents CH₂, or WP¹ represents NP¹, YP¹ represents CHR⁷ and XP¹ represents CH₂, may be prepared using established procedures. For example, compounds of formula (VII) as defined above may be prepared by Beckmann rearrangement of oxime sulfonates derived from the appropriately substituted dihydronaphthalenone with simultaneous nucleophilic trapping of the Intermediary iminocarbocation by organoaluminium reagents (Ref. J. Amer. Chem. Soc. 1983, 105, 2831-2843).

Compounds of formula (V), where L₁ is a hydroxyl group, may be prepared by reacting compounds of formula (VII) with compounds of formula (VII):

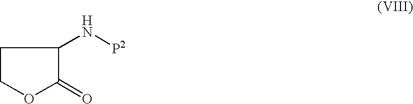

wherein P² represents a protecting group. The reaction is conveniently carried out by addition of a suitable activating agent, e.g. trimethylaluminium, to compounds of formula (VII) in a suitable solvent, e.g. DCM, under an inert atmosphere, e.g. nitrogen, suitably at room temperature followed by addition of a compound of formula (VIII) in a compatible solvent, e.g. DCM.

Compounds of formula (VIII) are known in the art or may be prepared from compounds of formula (IX) where HA is a suitable acid, e.g. hydrochloric acid, using methods well known to those skilled in the art. See, for example, “Protective groups in organic synthesis” by T. W. Greene and P. G. M. Wuts (John Wiley & sons 1991) or “Protecting Groups” by P. J. Kocienski (Georg Thieme Verlag 1994).

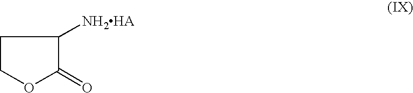

Compounds of formula (IX) are known compounds or may be prepared by methods known in the literature or processes known to those skilled in the art.

Compounds of formula (VII) where XP¹ represents —NP¹— and WP¹ and YP¹ represent —CH(R⁷)— may be prepared from compounds of formula (X):

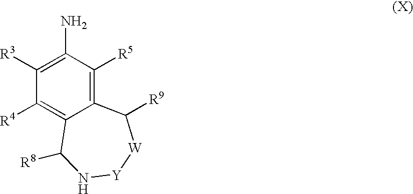

by protection with a suitable amine protecting group under standard conditions. For example,

where P¹ represents Boc, by treatment with di-tert butyl carboxylate (Boc₂O) in the presence of a suitable base, e.g. triethylamine (Et₃N), and in a suitable solvent, e.g. DCM or dioxane.

Compounds of formula (X) where W and Y represent —CH(R⁷)— and R⁸ represents hydrogen may be prepared from compounds of formula (XI):

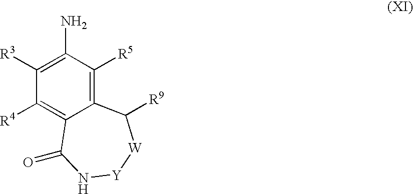

by reduction with a hydride source, e.g. borane in a suitable solvent, e.g. THF, suitably at reflux.

Compounds of formula (XI) where W and Y represent —CH(R⁷)— may be prepared from compounds of formula (XII):

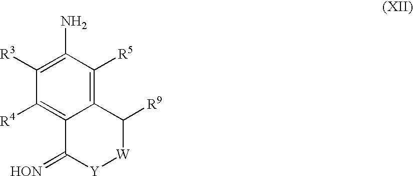

by rearrangement of the ketoxime to the corresponding amide in the presence of an acid catalyst, for example in the presence of polyphosphoric acid (PPA), suitably at elevated temperature such as 100 to 150° C.

Compounds of formula (XII) where W and Y represent —CH(R⁷)— may be prepared from compounds of formula (XIII):

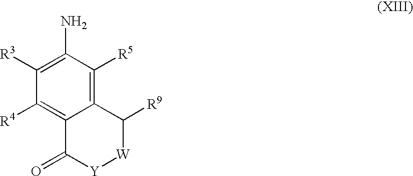

by treatment with a hydroxylamine salt, for example hydroxylamine hydrochloride, in the presence of a suitable base, for example sodium acetate, in a suitable solvent, for example aqueous EtOH.

Compounds of formula (XIII) are known compounds or may be prepared by methods known in the literature or processes known to those skilled in the art.

Compounds of formula (X) where W and Y represent —CH(R⁷)— and R⁸ represents C₁₋₄alkyl may be prepared from compounds of formula (XIV) where R⁸ represents C₁₋₄alkyl:

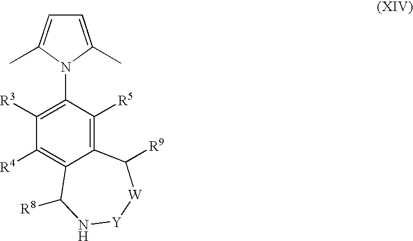

by reaction with hydroxylamine hydrochloride under standard conditions for example in the presence of an inorganic base e.g. potassium hydroxide in aqueous ethanol suitably at reflux.

Compounds of formula (XIV) where R⁸ represents C₁₋₄alkyl, may be prepared from compounds of formula (XV) where R⁸ represents hydrogen:

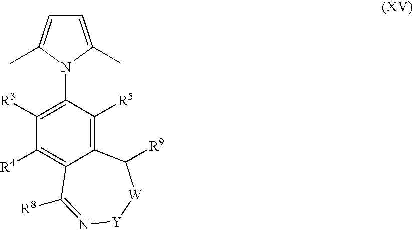

by reaction with an alkylmagnesium halide, e.g. methyl magnesium bromide, in the presence of a Lewis acid e.g. boron trifluoride etherate in a suitable solvent e.g. THF at −78° C. to room temperature. The reaction may be carried out with isolation of the Lewis acid complex (XVI). Compounds of formula (XV) may be prepared from compounds of formula (XVI) by reaction with the corresponding alkylmagnesium halide, e.g. methyl magnesium bromide, in a suitable solvent e.g. THF suitably at elevated temperature e.g. 50-70° C.

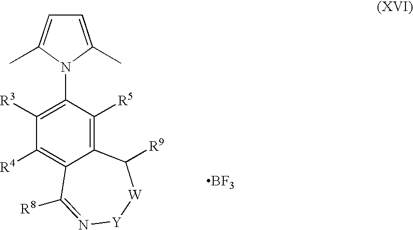

Compounds of formula (XVI) where R⁸ represents hydrogen may be prepared from compounds of formula (XVII) where R⁸ represents hydrogen:

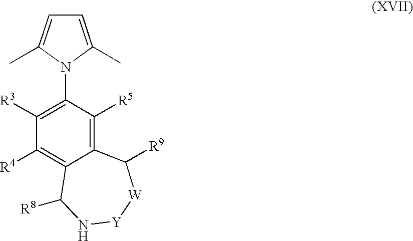

by oxidation, for example using manganese dioxide in a suitable solvent, e.g. DCM, suitably at room temperature.

Compounds of formula (XVII) where R⁸ represents hydrogen may be prepared from compounds of formula (XVIII) where R⁸ represents hydrogen:

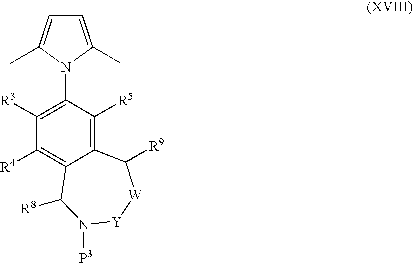

and P³ represents an amine protecting group e.g. trifluoroacetyl, by removal of the group P³ under standard conditions. For example when P³ represents trifluoroacetyl, removal of the protecting group may be effected under basic conditions, for example using potassium carbonate in aqueous methanol suitably at reflux.

Compounds of formula (XVIII) where R⁸ represents hydrogen may be prepared from compounds of formula (X) where R⁸ represents hydrogen by protection with a suitable amine protecting group P³ under standard conditions, followed by reaction with 2,5-hexanedione in the presence of an organic acid, e.g. 4-toluenesulfonic acid hydrate, in a suitable solvent, e.g. toluene, suitably at reflux with removal of water, for example in a Dean-Stark separator.

It will be appreciated by those skilled in the art that compounds of formula (I) or a solvate thereof may be synthesized from appropriate Intermediates via solid phase chemistry processes.

Compounds of formula (VII) where WP¹ represents —NP¹— and XP¹ and YP¹ represent —CH(R⁷)— may be prepared from compounds of formula (XIX):

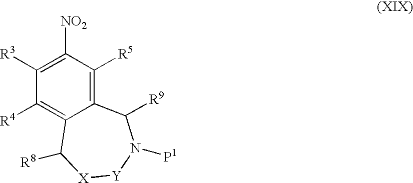

by reaction with hydrogen in the presence of a metal catalyst, for example, palladium/C, in a suitable solvent, e.g. EtOH, suitably at room temperature.

Compounds of formula (XIX) may be prepared from compounds of formula (XX):

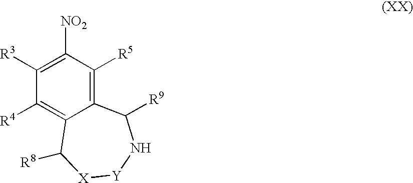

by protection with a suitable amine protecting group under standard conditions. For example,

where P¹ represents Boc, by treatment with di-tert butyl carboxylate (Boc₂O) in the presence of a suitable base, e.g. triethylamine (Et₃N), and in a suitable solvent, e.g. DCM.

Compounds of formula (XX) are known compounds or may be prepared by methods known in the literature or processes known to those skilled in the art.

Compounds of formula (VII) where WP¹ represents —NP¹— and XP¹ and YP¹ represent —CH(R⁷)— are known compounds or may be prepared by methods known in the literature or processes known to those skilled in the art.

Compounds of formula (I) where R² is a substituent other than hydrogen may be prepared by reacting a compound of formula (I) with a P¹ protecting group as appropriate where R² is hydrogen with a compound of formula (XXI):

R²-D (XXI)

wherein P¹ and R² are defined as above and D is a suitable leaving group such as a halide, e.g. bromide or iodide, followed by removal of the protecting group P¹ as appropriate. The reaction is effected in a suitable organic solvent, e.g. THF, DMF (N,N-dimethylformamide), MeCN in the presence of a base, e.g. LiHMDS (lithium hexamethyldisilylamide), potassium carbonate or sodium carbonate, at a temperature range from −78° C. to +50° C., suitably room temperature to 40° C. Furthermore, it will appreciated that the substituent R², other than hydrogen, may be introduced at various Intermediate stages by methods well known to those skilled in the art.

It will be appreciated by persons skilled in the art that compounds of formula (I) may be prepared by interconversion, utilising other compounds of formula (I), which are optionally protected by standard protecting groups, as precursors. For instance, compounds of formula (I) where X, Y or W is —N(R⁶) and R⁶ is hydrogen, may be converted into compounds of formula (I) where X, Y or W is —N(R⁶) and R⁶ is C₁₋₄alkyl by alkylation. For example, by treatment with paraformaldehyde under acidic conditions, e.g. formic acid, in a suitable solvent e.g. chloroform, suitably under reflux. Alternatively, alkylation may be carried out by treatment with tetramethylammonium triacetoxyborohydride under acidic conditions, e.g. acetic acid, in a suitable solvent e.g. acetone.

The various general methods described above may be useful for the introduction of the desired groups at any stage in the stepwise formation of the required compound, and it will be appreciated that these general methods can be combined in different ways in such multi-stage processes. The sequence of the reactions in multi-stage processes should of course be chosen so that the reaction conditions used do not affect groups in the molecule which are desired in the final product. For example, those skilled in the art will appreciate that, alkylation of X, Y or W may also be carried out on compounds of formula (IV) by removal of the protecting group and selective alkylation as described above, or at other convenient stages. Alternatively, compounds of formula (I) where R² represents —C₁₋₃alkylCO₂C₁₋₄alkyl may be converted to compounds of formula (I) where R² represents —C₁₋₃alkylCO₂H under acidic conditions, or to other compounds of formula (I) where R² represents —C₁₋₃alkylCO₂C₁₋₄ alkyl by transesterification under acidic conditions with the appropriate alcohol (C₁₋₄alkylOH).

Those skilled in the art will appreciate that in the preparation of compounds of formula (I) and pharmaceutically acceptable derivative(s) thereof it may be beneficial to protect certain stages from light.

Those skilled in the art will appreciate that in the preparation of compounds of formula (I) and/or solvates thereof it may be necessary and/or desirable to protect one or more sensitive groups in the molecule or the appropriate Intermediate to prevent undesirable side reactions. Suitable protecting groups for use according to the present invention are well known to those skilled in the art and may be used in a conventional manner. See, for example, “Protective groups in organic synthesis” by T. W. Greene and P. G. M. Wuts (John Wiley & sons 1991) or “Protecting Groups” by P. J. Kocienski (Georg Thieme Verlag 1994). Examples of suitable amino protecting groups include acyl type protecting groups (e.g. formyl, trifluoroacetyl, acetyl), aromatic urethane type protecting groups (e.g. benzyloxycarbonyl (Cbz) and substituted Cbz), aliphatic urethane protecting groups (e.g. 9-fluorenylmethoxycarbonyl (Fmoc), t-butyloxycarbonyl (Boc), isopropyloxycarbonyl, cyclohexyloxycarbonyl) and alkyl or aralkyl type protecting groups (e.g. benzyl, trityl, chlorotrityl).

Various Intermediate compounds used in the above-mentioned process, including but not limited to certain compounds of formulae (II), (IV) and (V) constitute a further aspect of the present invention.

The present invention will now be further illustrated by the accompanying examples which should not be construed as limiting the scope of the invention in any way.

All publications, including but not limited to patents and patent applications, cited in this specification are herein incorporated by reference as if each individual publication were specifically and individually indicated to be incorporated by reference herein as though fully set forth.

### EXAMPLES

### Abbreviations

| | |
|---|---|
| MeCN | Acetonitrile |
| EtOH | Ethanol |
| MeOH | Methanol |
| DCM | Dichloromethane |
| DMF | N,N-dimethyiformamide |
| THF | tetrahydrofuran |
| HATU | O-(7-Azabenzotriazol-1-yl)-N,N,N′,N′- |
| | tetramethyluronium-hexafluoro-phosphate |
| min | minutes |
| h | hours |
| m | multiplet |
| s | singlet |
| d | doublet |
| t | triplet |
| dd | double doublet |
| br. s | broad singlet |

### Intermediate 1

### (1E)-6-Amino-3,4-dihydro-1(2H)-naphthalenone oxime

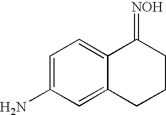

A mixture of 6-amino-3,4-dihydro-1(2H)-naphthalenone (32 g, 0.199 mol), hydroxylamine hydrochloride (20.71 g, 0.298 mol) and sodium acetate (24.44 g, 0.298 mol) in EtOH (480 ml) and water (192 ml) was heated to 90° C., under nitrogen, for 3 h. The mixture was cooled to room temperature and evaporated under reduced pressure. The residue was diluted with water (150 ml) and the mixture was allowed to stand at room temperature for 72 h. The precipitate was collected by filtration, washed with water and vacuum dried for 24 h to give the title compound as a brown solid (34.6 g).

Mass spectrum: Found: MH⁺ 177

H.p.l.c. Rₜ 2.11 min

### Intermediate 2

### 7-Amino-2,3,4,5-tetrahydro-1H-2-benzazepin-1-one

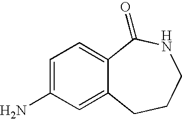

Polyphosphoric acid (488 g) was heated to 90° C. in a 2 litre flask equipped with a mechanical stirrer. (1E)-6-Amino-3,4-dihydro-1(2H)-naphthalenone oxime (see Intermediate 1) (34.6 g, 0.196 mol) was added in portions over 1 h with rapid stirring. The temperature was increased to 110° C. and the mixture was heated for 1.5 h. As a fine solid suspension was still present, the mixture was heated at 120° C. for a further 2 h and then poured onto iced water (1500 ml). The gum was broken up and stirred for 30 min and then basified by the careful addition of 10M aqueous sodium hydroxide solution (1300 ml) keeping the temperature below 90° C. 10 g of solid sodium hydroxide was added and the mixture was cooled in an ice bath and a solid precipitated. Around one third of the mixture was filtered through a sinter funnel. The solid was re-suspended in a mixture of DCM and water and re-filtered. This was repeated until a total of 4000 ml (1:1) DCM:water had been used. The remaining two thirds of material was poured onto a much larger sinter funnel and a similar extraction and filtration method was used once more collecting a further 4000 ml of 1:1 DCM:water. The mixture was left to stand for 16 h. Since some solid had filtered through, the mixtures were refiltered. This mixture was filtered (×3) as more solid continued to precipitate. The layers were separated and the aqueous was washed with DCM (1000 ml). The organic extracts were combined and evaporated to give the title compound as a brown solid (27 g, contains 4% 7-amino-1,3,4,5-tetrahydro-2H-1-benzazepin-2-one).

Mass spectrum: Found: MH⁺ 177

H.p.l.c. Rₜ 1.51 min

### Intermediate 3

### 2,3,4,5-Tetrahydro-1H-2-benzazepin-7-amine

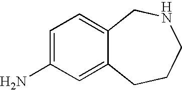

A suspension of 7-amino-2,3,4,5-tetrahydro-1H-2-benzazepin-1-one (see Intermediate 2) (27 g, 0153 mol) in anhydrous THF (888 ml) was stirred at room temperature under nitrogen and treated with a 1M solution of borane in THF (800 ml) dropwise over 1.5 h. Once the addition was complete the mixture was heated at reflux for 6 h and then allowed to stand for 2 days at room temperature. The reaction mixture was then heated at reflux for a further 16 h and cooled to room temperature. MeOH (444 ml) was slowly added and then the mixture was evaporated to dryness. The white solid was dissolved in MeOH (600 ml) and 5M hydrochloric acid (480 ml) was slowly added. The reaction mixture was heated at reflux for 2 h and cooled to room temperature overnight. The mixture was basified with 10M aqueous sodium hydroxide solution to pH>13, evaporated to remove volatiles and extracted with DCM (3×600 ml). The organic extracts were combined, washed with brine (300 ml), passed through a hydrophobic frit and evaporated under reduced pressure to give a dark brown oil. This oil was purified in 3 batches using a CombiFlash® Companion®. 3 g was purified on a 120 g Redisep® silica column and the remainder on 2×330 g Redisep® silica columns eluting with a DCM:methanolic ammonia gradient to give the title compound as a dark orange oil (13.11 g).

¹H NMR (CDCl₃) δ: 1.73 (2H, m), 2.1 (br. s, 2H+water), 2.84 (2H, m), 3.19 (2H, t), 3.60 (1H, br. s), 3.87 (2H, s), 6.43 91H, m), 6.52 (1H, m), 6.92 (1H, d).

### Intermediate 4

### 1,1-Dimethylethyl 7-amino-1,3,4,5-tetrahydro-2H-2-benzazepine-2-carboxylate

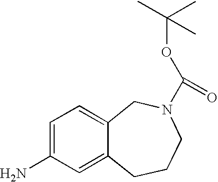

A solution of 2,3,4,5-tetrahydro-1H-2-benzazepin-7-amine (see Intermediate 3) (13.05 g, 80.43 mmol) in dioxane (600 ml) and water (56 ml) (two preparations of 2,3,4,5-tetrahydro-1H-2-benzazepin-7-amine were combined with warming) was cooled to 10° C. and treated with di-tert-butyl dicarbonate (15.7 g, 71.93 mmol) in portions over 15 min with the last portion being added in dioxane (10 ml). The resulting solution was stirred in the cooling bath for 20 h during which time it reached room temperature*. The reaction mixture was evaporated under reduced pressure and the residue was partitioned between water (200 ml) and DCM (400 ml). The layers were separated and the aqueous layer was extracted with DCM (200 ml). The organic extracts were combined, washed with brine, dried (anhydrous sodium sulfate), filtered twice (a fine precipitate came through after the first filtration) and evaporated to dryness. The product was dissolved in DCM and purified on a CombiFlash® Companion® using a 330 g Redisep® silica column eluting with 5% to 60% ethyl acetate in cyclohexane gradient. The fractions were combined and evaporated to dryness. The gum was taken up in DCM and re-evaporated to give the title compound as a pale cream partial foam (13.20 g).

Mass spectrum: Found: MH⁺ 263.

H.p.l.c. Rₜ=2.64 min. *Note: No sign of other regioisomer. However, bis-boccing has gone further on. In future do not let it reach room temperature.

### Intermediate 5

### 1,1-Dimethylethyl 7-[(N-{[(phenylmethyl)oxy]carbonyl}-L-methionyl)amino]-1,3,4,5-tetrahydro-2H-2-benzazepine-2-carboxylate

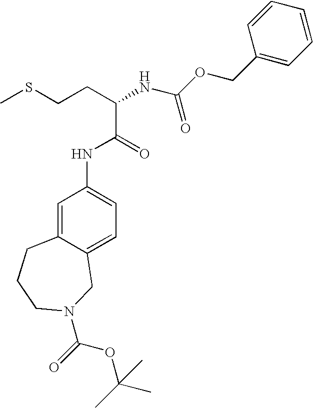

A solution of 1,1-dimethylethyl 7-amino-1,3,4,5-tetrahydro-2H-2-benzazepine-2-carboxylate (see Intermediate 4) (13.2 g, 50.33 mmol) in anhydrous DCM (600 ml) was stirred at room temperature under nitrogen. N-{[(Phenylmethyl)oxy]carbonyl}-L-methionine (14.26 g, 50.33 mmol) was added and the resulting mixture was stirred for 5 min until a solution formed. HATU (19.13 g, 50.31 mmol) was added and after 5 min N,N-diisopropylethylamine (9.6 ml, 55.1 mmol) was added dropwise over 2 min. The flask was stoppered and the reaction mixture was stirred at room temperature for 63 h. The bright orange light suspension was treated with saturated aqueous ammonium chloride solution (150 ml) and stirred for 10 min. The layers were separated and the aqueous phase was extracted with DCM (100 ml). The organic extracts were combined and washed with saturated aqueous NaHCO₃ solution (200 ml) and brine (200 ml), passed through a hydrophobic frit and evaporated to an orange gum. This was dissolved in DCM and purified on a CombiFlash® Companions using 2×330 g Redisep® silica columns eluting with a 15 to 60% ethyl acetate in cyclohexane gradient system to give a sticky white foam which was dried on a vacuum line for a short while to give the title compound as a sticky colourless foam (25.03 g).

Mass spectrum: Found: [M-H]-526.

H.p.l.c. Rₜ=3.62 min.

### Intermediate 6

### [(3S)-4-[(2-{[(1,1-Dimethylethyl)oxy]carbonyl}-2,3,4,5-tetrahydro-1H-2-benzazepin-7-yl)amino]-4-oxo-3-({[(Phenylmethyl)oxy]carbonyl}amino)butyl](dimethyl)sulfonium iodide

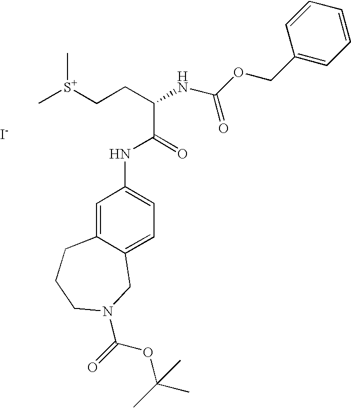

1,1-Dimethylethyl 7-[(N-{[(phenylmethyl)oxy]carbonyl}-L-methionyl)amino]-1,3,4,5-tetrahydro-2H-2-benzazepine-2-carboxylate (see Intermediate 5) (25.01 g, 47.39 mmol) was dissolved in dry MeCN (350 ml) under nitrogen. Iodomethane (30 ml, 482 mmol) was added and the solution stirred at room temperature for 5 min. The mixture was then allowed to stand at room temperature overnight in a stoppered flask. The mixture was then concentrated under reduced pressure to afford the title compound (31.75 g) as a yellow foam.

H.p.l.c. Rₜ=2.68 min.

### Intermediate 7

### 1,1-Dimethylethyl 7-[(3S)-2-oxo-3-({[(phenylmethyl)oxy]carbonyl}amino)-1-pyrrolidinyl]-1,3,4,5-tetrahydro-2H-2-benzazepine-2-carboxylate

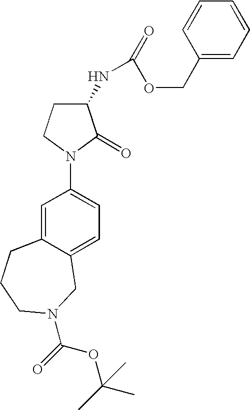

[(3S)-4-[(2-{[(1,1-Dimethylethyl)oxy]carbonyl}-2,3,4,5-tetrahydro-1H-2-benzazepin-7-yl)amino]-4-oxo-3-({[(phenylmethyl)oxy]carbonyl}amino)butyl](dimethyl)sulfonium iodide (see Intermediate 6) (31.75 g, 47.39 mmol) was dissolved in anhydrous MeCN (350 ml) at room temperature under nitrogen. Cesium carbonate (16.2 g, 49.72 mmol) was added and the resultant suspension was stirred vigorously at 60° C. for 70 min. On cooling, the mixture was treated with saturated aqueous ammonium chloride (50 ml) and evaporated under reduced pressure. The residue was partitioned between water (200 ml) and DCM (250 ml). The aqueous layer was then extracted with DCM (200 ml) and the combined organic phases were washed with brine (100 ml), passed through a hydrophobic frit and evaporated under reduced pressure to afford a pale yellow gelatinous solid. This was dissolved in DCM (50 ml) and purified on a CombiFlash® Companions using 2×330 g Redisep® silica columns eluting with a 20 to 80% ethyl acetate in cyclohexane gradient system, the fractions were eluted with slightly differing gradients and then the combined portions were evaporated under reduced pressure and dried in vacuo to give the title compound (21.58 g) as a white waxy solid.

Mass spectrum: Found: MH⁺ 480

H.p.l.c. Rₜ=3.36 min.

### Intermediate 8

### 1,1-Dimethylethyl 7-[(3S)-3-amino-2-oxo-1-pyrrolidinyl]-1,3,4,5-tetrahydro-2H-2-benzazepine-2-carboxylate

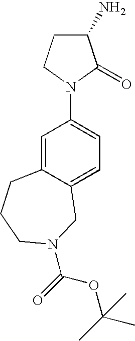

1,1-Dimethylethyl 7-[(3S)-2-oxo-3-({[(phenylmethyl)oxy]carbonyl}amino)-1-pyrrolidinyl]-1,3,4,5-tetrahydro-2H-2-benzazepine-2-carboxylate (see Intermediate 7) (21.48 g, 44.8 mmol) was stirred at room temperature in EtOH (550 ml) for 1 h during which time most of the material dissolved. The mixture was added to wet palladium hydroxide on carbon (4.19 g, 20% palladium by weight, E101NE/W), washing in with further EtOH (50 ml). The mixture was then stirred under an atmosphere of hydrogen for 15.5 h. The catalyst was then removed by filtration through a flash fibre filter and washed with a small amount of ethanol. The combined filtrate and washings were evaporated under reduced pressure and dried in vacuo at room temperature to afford the title compound (13.21 g) as a white foam.

Mass spectrum: Found: [MNH₄]⁺ 363

H.p.l.c. Rₜ=2.17 min.

### Intermediate 9

### 1,1-Dimethylethyl 7-[(3S)-3-({[(E)-2-(5-chloro-2-thienyl)ethenyl]sulfonyl}amino)-2-oxo-1-pyrrolidinyl]-1,3,4,5-tetrahydro-2H-2-benzazepine-2-carboxylate

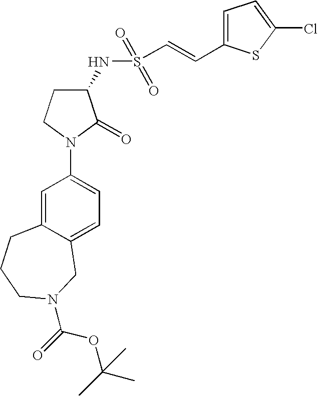

1,1-Dimethylethyl 7-[(3S)-3-amino-2-oxo-1-pyrrolidinyl]-1,3,4,5-tetrahydro-2H-2-benzazepine-2-carboxylate (see Intermediate 8) (13.17 g, 38.1 mmol) was dissolved in anhydrous MeCN (600 ml) at room temperature under nitrogen. Anhydrous pyridine (9.25 ml, 114.3 mmol) was added to the solution and stirred for 7 min before portionwise addition of (E)-2-(5-chloro-2-thienyl)ethanesulfonyl chloride (11.12 g, 45.73 mmol) over 12 min. The suspension was then stirred at room temperature for 18.5 h before evaporation under reduced pressure. The residue was partitioned between DCM (450 ml) and 0.5M hydrochloric acid (500 ml). The layers were separated and the aqueous phase extracted with DCM (200 ml). The combined organic phases were washed sequentially with saturated aqueous sodium bicarbonate (250 ml) and brine (250 ml) before passing through a hydrophobic frit and evaporating under reduced pressure to afford a yellow solid. This solid was stirred in diethyl ether (560 ml) for 1 h then collected by filtration, washed with ether and dried in vacuo at 50° C. for 1 h to afford the title compound (18.27 g) as a yellow solid.

Mass spectrum: Found: [MNH₄]⁺ 569, 571

H.p.l.c. Rₜ=3.71 min.

### Intermediate 10

### 6-Bromo-1-[tris(1-methylethyl)silyl]-1H-indole

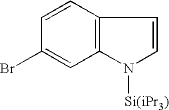

A solution of 6-bromo-1H-indole (2.0 g, 0.01 mmol) in dry THF (20 ml) at 0° C. was treated with sodium hydride (60% dispersion in mineral oil; 450 mg, 0.012 mmol), in portions, stirring for 30 min. Chloro[tris(1-methylethyl)]silane (2.57 ml, 0.012 mmol) was added to the reaction in a drop-wise manner, allowing the reaction to warm up to ambient temperature and stirring for 18 h. The reaction was concentrated under reduced pressure, partitioning the residue between DCM and saturated aqueous sodium bicarbonate solution. The separated organic phase was passed through a hydrophobic frit and re-concentrated to a small volume before being loaded onto a pre-conditioned silica phase SPE column (150 ml/70 g) eluting with cyclohexane:ethyl acetate (0-2%). Fractions containing product were combined and concentrated under reduced pressure to give the title compound (3.51 g) as colourless oil.

Mass spectrum: Found: MH⁺ 352/354

H.p.l.c. Rₜ 4.51 min

### Intermediate 11

### 3-Chloro-1-[tris(1-methylethyl)silyl]-1H-indole-6-sulfonyl chloride

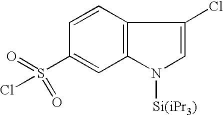

A solution of 6-bromo-1-[tris(1-methylethyl)silyl]-1H-indole (see Intermediate 10) (2.30 g, 6.53 mmol) in dry THF (45 ml) at −78° C. was treated with n-butyllithium (1.6M in hexanes; 4.29 ml, 6.86 mmol) in a drop-wise manner. The mixture was allowed to stir for 1 h and then poured into a stirred solution of sulfuryl chloride (1.22 ml, 0.0152 mmol in dry cyclohexane (45 ml)) at 0° C. The reaction was allowed to warm up to ambient temperature and stirred for 2.5 h, then quenched with water (45 ml) stirring for 30 min. The separated organic layer was passed through a hydrophobic frit and concentrated under reduced pressure. The crude material was dissolved in cyclohexane and loaded onto a pre-conditioned silica phase SPE column (150 ml/70 g) eluting with cyclohexane:ethyl acetate (0-10%). Fractions containing product were combined and concentrated under reduced pressure to give the title compound (1.18 g) as a beige gum.

### Following Dimethylamine Quench:

Mass spectrum: Found: MH⁺ 415

H.p.l.c. Rₜ 4.22 min

Also identified was the deprotected product 3-chloro-1H-indole-6-sulfonyl chloride.

### Following Dimethylamine Quench:

Mass spectrum: Found: MH⁺ 259

H.p.l.c. Rₜ 3.03 min

### Intermediate 12

### 1,1-Dimethylethyl 7-((3S)-3-[({(3-chloro-1H-indol-6-yl)sulfonyl]amino}-2-oxo-1-pyrrolidinyl)-1,3,4,5-tetrahydro-2H-2-benzazepine-2-carboxylate

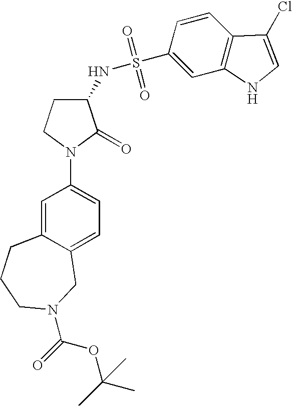

Prepared from 1,1-dimethylethyl 7-[(3S)-3-amino-2-oxo-1-pyrrolidinyl]-1,3,4,5-tetrahydro-2H-2-benzazepine-2-carboxylate (see Intermediate 8) and 3-chloro-1-[tris(1-methylethyl)silyl]-1H-indole-6-sulfonyl chloride (see Intermediate 11) using a similar procedure to that described for Intermediate 9. This afforded a mixture of Indole N—H and N-tri-isopropylsilyl material which was separated using a CombiFlash® Companions with Redisep® silica columns eluting with a 30 to 100% ethyl acetate in cyclohexane gradient system to afford the title compound.

Mass spectrum: Found: MH⁺ 559, 561

H.p.l.c. Rₜ 3.52 min

Isolated N-tri-isopropylsilyl material was transformed into the N—H compound via the following procedure:

1,1-Dimethylethyl 7-{(3S)-3-[({3-chloro-1-[tris(1-methylethyl)silyl]-1H-indol-6-yl}sulfonyl)amino]-2-oxo-1-pyrrolidinyl}-1,3,4,5-tetrahydro-2H-2-benzazepine-2-carboxylate (160 mg, 0.22 mmol) and tetraethylammonium fluoride (50 mg, 0.33 mmol) were added to THF (10 ml) under nitrogen. Acetic acid (0.2 ml) was added and the reaction mixture stirred at room temperature for 1 h. Saturated aqueous ammonium chloride was added and the mixture evaporated under reduced pressure. The residue was partitioned between water (10 ml) and chloroform (10 ml). The layers were separated using a hydrophobic frit and the organic phase evaporated under reduced pressure. The residue was purified using a 10 g silica SPE cartridge eluting with chloroform then 1:1 chloroform:ethyl acetate then ethyl acetate to afford the title compound (115 mg).

Mass spectrum: Found: MH⁺ 559, 561

H.p.l.c. Rₜ 3.52 min

### Intermediate 13

### 1,1-Dimethylethyl 7-[(3S)-3-amino-2-oxo-1-pyrrolidinyl]-1,2,4,5-tetrahydro-3H-3-benzazepine-3-carboxylate

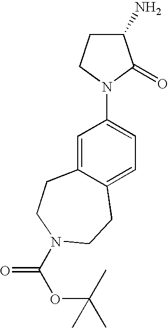

Prepared from 1,1-dimethylethyl 7-amino-1,2,4,5-tetrahydro-3H-3-benzazepine-3-carboxylate (EP284384) using a similar procedure to that described for Intermediate 8.

Mass spectrum: Found: MH⁺ 346, [MNH₄]⁺ 363

H.p.l.c. Rₜ 2.53 min

### Intermediate 14

### 1,1-Dimethylethyl 7-((3S)-3-{[(3-chloro-1H-indol-6-yl)sulfonyl]-amino}-2-oxo-1-pyrrolidinyl)-1,2,4,5-tetrahydro-3H-3-benzazepine-3-carboxylate

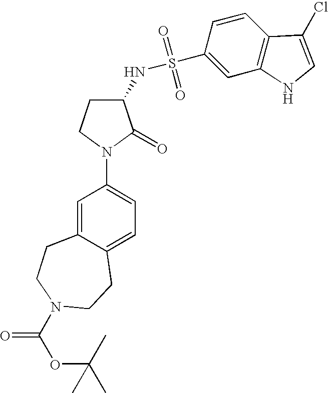

Prepared from 1,1-dimethylethyl 7-[(3S)-3-amino-2-oxo-1-pyrrolidinyl]-1,2,4,5-tetrahydro-3H-3-benzazepine-3-carboxylate (see Intermediate 13) and 3-chloro-1-[tris(1-methylethyl)silyl]-1H-indole-6-sulfonyl chloride (see Intermediate 11) using a similar procedure to that described for Intermediate 9. This afforded a mixture of Indole N—H and N-tri-isopropylsilyl material which was processed using a similar procedure to that described for Intermediate 12.

Mass spectrum: Found: MNH₄⁺ 576, 578

H.p.l.c. Rₜ 3.71 min

### Intermediate 15

### 1,1-Dimethylethyl 8-nitro-1,3,4,5-tetrahydro-2H-2-benzazepine-2-carboxylate

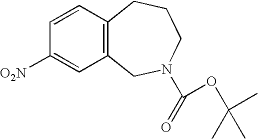

To a mixture of 8-nitro-2,3,4,5-tetrahydro-1H-2-benzazepine (Bioorg. Med. Chem., 2001, 9, 1957-1965) (16.02 g, 83.2 mmol) and di-tert-butyl dicarbonate (20 g, 88.8 mmol) in DCM (600 ml) was added triethylamine (12.5 ml) and the resulting mixture was stirred at ambient temperature under nitrogen for 3 days. The mixture was washed with water (3×150 ml) and saturated brine (100 ml), then passed through a hydrophobic frit and concentrated in vacuo. The resultant yellow-brown gum was dissolved/suspended in cyclohexane:ethyl acetate (5:1; 150 ml), then loaded onto a 400 g Biotage™ silica cartridge and eluted with cyclohexane:ethyl acetate (5:1). Appropriate fractions were combined and evaporated to afford the title compound as a pale yellow solid (3.99 g) after drying in vacuo.

Mass spectrum: [MNH₄]⁺ 310

H.p.l.c. Rₜ=3.34 min.

### Intermediate 16

### 1,1-Dimethylethyl 8-amino-1,3,4,5-tetrahydro-2H-2-benzazepine-2-carboxylate

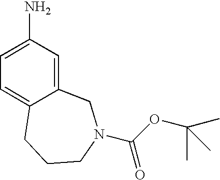

A solution of 1,1-dimethylethyl 8-nitro-1,3,4,5-tetrahydro-2H-2-benzazepine-2-carboxylate (see Intermediate 15) (3.98 g, 13.6 mmol) in EtOH (140 ml) was added to moist 10% palladium on carbon (2 g, E101 NE/N) and the mixture was then stirred under an atmosphere of hydrogen for 1.75 h. The catalyst was then removed by filtration under nitrogen, moistened with aqueous sodium bisulphate and the solution evaporated in vacuo to afford the title compound (3.37 g) as a pale grey solid.

Mass spectrum: MH⁺ 263

H.p.l.c. Rₜ=2.54 min

### Intermediate 17

### 1,1-Dimethylethyl 8-[(3S)-3-amino-2-oxo-1-pyrrolidinyl]-1,3,4,5-tetrahydro-2H-2-benzazepine-2-carboxylate

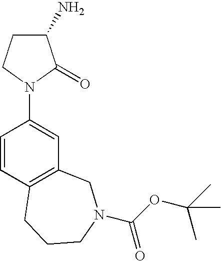

Prepared from 1,1-dimethylethyl 8-amino-1,3,4,5-tetrahydro-2H-2-benzazepine-2-carboxylate (see Intermediate 16) by a procedure similar to those described for Intermediates 5, 6, 7 and 8.

Mass spectrum: Found: MH⁺ 346

H.p.l.c. Rₜ=2.29 min.

### Intermediate 18

### 1,1-Dimethylethyl 8-[(3S)-3-({[(E)-2-(5-chloro-2-thienyl)ethenyl]sulfonyl}amino)-2-oxo-1-pyrrolidinyl]-1,3,4,5-tetrahydro-2H-2-benzazepine-2-carboxylate

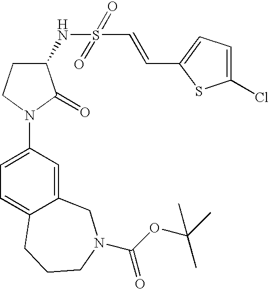

Prepared from 1,1-dimethylethyl 8-[(3S)-3-amino-2-oxo-1-pyrrolidinyl]-1,3,4,5-tetrahydro-2H-2-benzazepine-2-carboxylate (see Intermediate 17) (30 ml, 804 mg, 2.37 mmol) and (E)-2-(5-chloro-2-thienyl)ethanesulfonyl chloride (681 mg, 2.8 mmol) by a procedure similar to that described for Intermediate 9 followed by purification on a 20 g Redisep® silica cartridge eluting with DCM:ethyl acetate (6:1).

Mass spectrum: Found: [MNH₄]⁺ 569, 571

H.p.l.c. Rₜ=3.59 min.

### Intermediate 19

### 1,1-Dimethylethyl 7-{(3S)-3-[{[(E)-2-(5-chloro-2-thienyl)ethenyl]sulfonyl}(methyl)amino]-2-oxo-1-pyrrolidinyl}-1,3,4,5-tetrahydro-2H-2-benzazepine-2-carboxylate

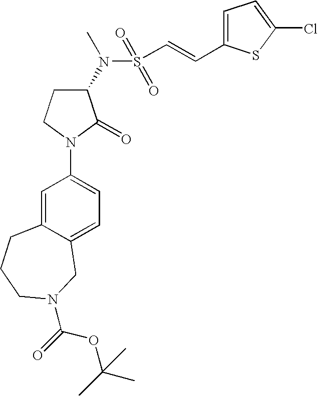

1,1-Dimethylethyl 7-[(3S)-3-({[(E)-2-(5-chloro-2-thienyl)ethenyl]sulfonyl}amino)-2-oxo-1-pyrrolidinyl]-1,3,4,5-tetrahydro-2H-2-benzazepine-2-carboxylate (see Intermediate 9) (101 mg, 0.18 mmol) was dissolved in anhydrous DMF (2 ml) under nitrogen. Potassium carbonate (31.5 mg, 0.23 mmol) was then added, followed by iodomethane (22.8 μl, 0.36 mmol). The system was sealed, and the mixture was stirred at 40° C. for 65 h. Saturated aqueous ammonium chloride (1 ml) was added and the volatiles were removed under reduced pressure. The residue was partitioned between DCM (10 ml) and water (10 ml) and the organic phase was separated using a hydrophobic frit. The aqueous phase was extracted with DCM (10 ml). The organic extracts were combined and concentrated under reduced pressure and the crude product was purified by column chromatography on a 12 g Redisep® silica cartridge eluted with 15%-100% ethyl acetate in cyclohexane on an ISCO Companion® system. Appropriate fractions were combined and evaporated under reduced pressure to give the title compound (91 mg) as a white solid.

Mass spectrum: Found [MNH₄⁺]⁻ 583, 585

H.p.l.c. Rₜ 3.33 min

### Intermediate 20

### 1,1-Dimethylethyl 7-[(3S)-3-({[(E)-2-(5-chloro-2-thienyl)ethenyl]sulfonyl}{2-[(1,1-dimethylethyl)oxy]-2-oxoethyl}amino)-2-oxo-1-pyrrolidinyl]-1,3,4,5-tetrahydro-2H-2-benzazepine-2-carboxylate

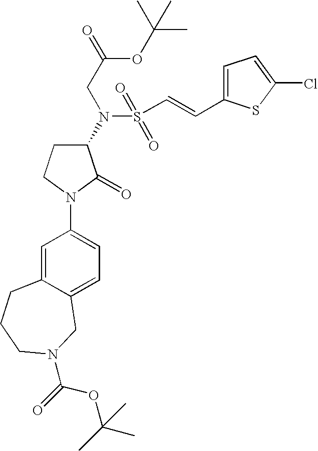

1,1-Dimethylethyl 7-[(3S)-3-({[(E)-2-(5-chloro-2-thienyl)ethenyl]sulfonyl}amino)-2-oxo-1-pyrrolidinyl]-1,3,4,5-tetrahydro-2H-2-benzazepine-2-carboxylate (see Intermediate 9) (311 mg, 0.56 mmol) was dissolved in anhydrous DMF (4 ml) under nitrogen. Potassium carbonate (92 mg, 0.66 mmol) was then added, followed by t-butyl bromoacetate (166.6 μl). The system was sealed, and the mixture was stirred at 40° C. for 41 h. Saturated aqueous ammonium chloride (1 ml) was added and the volatiles were removed under reduced pressure. The residue was partitioned between DCM (15 ml) and water (15 ml) and the organic phase was separated using a hydrophobic frit. The aqueous phase was extracted with DCM (15 ml). The organic extracts were combined and concentrated under reduced pressure and the crude product was purified by column chromatography on a 12 g Redisep® silica cartridge eluted with 10%-100% ethyl acetate in cyclohexane on an ISCO Companion® system. Appropriate fractions were combined and evaporated under reduced pressure to give the title compound (346 mg) as a white foam.

Mass spectrum: Found [MH⁺]⁻ 666, 668

H.p.l.c. Rₜ 3.96 min

### Intermediate 21

### 1,1-Dimethylethyl 7-{(3S)-3-[({[(E)-2-(5-chloro-2-thienyl)ethenyl]sulfonyl}(2-hydroxyethyl)amino]-2-oxo-1-pyrrolidinyl}-1,3,4,5-tetrahydro-2H-2-benzazepine-2-carboxylate

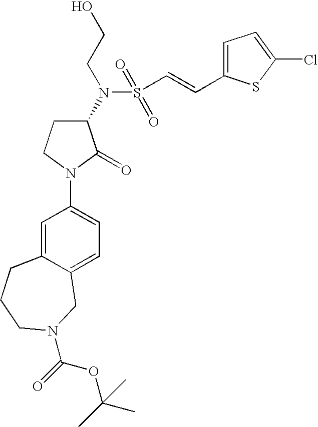

1,1-Dimethylethyl 7-[(3S)-3-({[(E)-2-(5-chloro-2-thienyl)ethenyl]sulfonyl}amino)-2-oxo-1-pyrrolidinyl]-1,3,4,5-tetrahydro-2H-2-benzazepine-2-carboxylate (see Intermediate 9) (102 mg, 0.18 mmol) was dissolved in anhydrous DMF (2 ml) under nitrogen. Potassium carbonate (32.5 mg, 0.23 mmol) was then added, followed by 2-bromoethanol (26.1 μl, 0.37 mmol). The system was sealed and the mixture was stirred at 40° C. for 25.25 h then allowed to stand at room temperature for 2 nights. Saturated aqueous ammonium chloride (1 ml) was added and the volatiles were removed under reduced pressure. The residue was partitioned between DCM (10 ml) and water (10 ml) and the organic phase was separated using a hydrophobic frit. The aqueous phase was extracted with DCM (10 ml). The organic extracts were combined and concentrated under reduced pressure and the crude product was purified by column chromatography on a 12 g Redisep® silica cartridge eluted with 10%-100% ethyl acetate in cyclohexane on an ISCO Companion® system. Appropriate fractions were combined and evaporated under reduced pressure to give the title compound (11 mg) as a white solid.

Mass spectrum: Found [MH⁺]⁻ 596, 598

H.p.l.c. Rₜ 3.60 min

### Intermediate 22

### 1,1-Dimethylethyl 7-[(3S)-3-({[(E)-2-(5-chloro-2-thienyl)ethenyl]sulfonyl}{2-[(1-methylethyl)oxy]-2-oxoethyl}amino)-2-oxo-1-pyrrolidinyl]-1,3,4,5-tetrahydro-2H-2-benzazepine-2-carboxylate

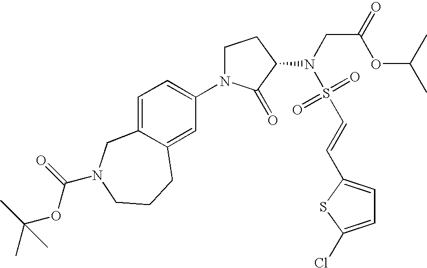

Prepared from 1,1-dimethylethyl 7-[(3S)-3-({[(E)-2-(5-chloro-2-thienyl)ethenyl]sulfonyl}amino)-2-oxo-1-pyrrolidinyl]-1,3,4,5-tetrahydro-2H-2-benzazepine-2-carboxylate (see Intermediate 9) and isopropyl bromoacetate using the a procedure similar to that described for Intermediate 20, using the quantities given below, followed by purification of the crude product by column chromatography on a 20 g Isolute silica cartridge eluting with ethyl acetate:cyclohexane mixtures (3:1 followed by 2:1).

1,1-dimethylethyl 7-[(3S)-3-({[(E)-2-(5-chloro-2-thienyl)ethenyl]sulfonyl}amino)-2-oxo-1-pyrrolidinyl]-1,3,4,5-tetrahydro-2H-2-benzazepine-2-carboxylate (0.81 mg, 1.36 mmol)_{—}

Potassium carbonate (225 mg, 1.63 mmol)

Isopropyl bromoacetate (0.35 ml, 2.7 mmol)

Mass spectrum: Found [MH⁺] 652, 654

H.p.l.c. Rₜ 3.88 min

### Intermediates 23 and 24

### 1,1-Dimethylethyl 7-((3S)-3-{{[(E)-2-(5-chloro-2-thienyl)ethenyl]sulfonyl}[2-(ethyloxy)-1-methyl-2-oxoethyl]amino}-2-oxo-1-pyrrolidinyl)-1,3,4,5-tetrahydro-2H-2-benzazepine-2-carboxylate (isomer 1 and isomer 2)

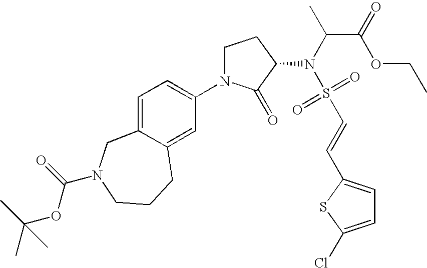

Prepared from 1,1-dimethylethyl 7-[(3S)-3-({[(E)-2-(5-chloro-2-thienyl)ethenyl]sulfonyl}amino)-2-oxo-1-pyrrolidinyl]-1,3,4,5-tetrahydro-2H-2-benzazepine-2-carboxylate (1.56 g, 2.7 mmol) (see Intermediate 9) and ethyl 2-bromopropionate (0.71 ml, 5.4 mmol) using a procedure similar to that described for Intermediate 20, using the quantities given below, followed by purification of the crude product by column chromatography on a 70 g Isolute silica cartridge eluting with ethyl acetate:cyclohexane mixtures (3:1 followed by 2:1). A portion (0.10 g) of the derived mixture of diastereomers (0.84 g) was separated on a chiralpak AD column eluting with 20% ethanol/heptane/0.1% trifluoroacetic acid to give the title compound (isomer 1) (0.035 g) and the title compound (isomer 2) (0.050 g) as white solids.

1,1-dimethylethyl 7-[(3S)-3-({[(E)-2-(5-chloro-2-thienyl)ethenyl]sulfonyl}amino)-2-oxo-1-pyrrolidinyl]-1,3,4,5-tetrahydro-2H-2-benzazepine-2-carboxylate (1.56 g, 2.7 mmol)

Potassium carbonate (450 mg, 3.25 mmol)

Ethyl 2-bromopropionate (0.71 ml, 5.4 mmol)

### Isomer 1:

Mass spectrum: Found [MH⁺] 652, 654

H.p.l.c. Rₜ 3.87 min

### Isomer 2:

Mass spectrum: Found [MH⁺] 652

H.p.l.c. Rₜ 3.86 min

### Intermediate 25

### 4-(2,3-Difluorophenyl)-3-butyn-1-ol

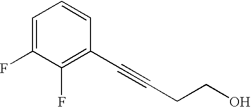

To a solution of 1-bromo-2,3-difluorobenzene (10.0 g, 51.82 mmol, Aldrich) in dimethylamine (200 ml) under nitrogen were added Bis(tripheylphenylphosphine)palladium(II) dichloride (0.73 g, 1.037 mmol), copper(I) iodide (0.1 g, 0.525 mmol) and 3-butyn-1-ol (11.76 ml). The temperature was raised from ambient to 65° C. and held there with stirring for 20 h, after which the mixture was cooled and all volatiles removed by rotary evaporation. The residue was partitioned between a mixture of 2N HCl (400 ml), brine (100 ml) and dichloromethane (400 ml). A second 100 ml dichloromethane extract and the layers were separated and aqueous was washed with DCM (100 ml). Extracts were combined and the mixture passed through a hydrophobic frit and evaporated under reduced pressure to give a dark brown oil. This oil was purified using a CombiFlash® Companion® 330 g Redisep® silica column eluting with a gradient of cyclohexane:ether (0 to 100%) affording the title compound as a light orange oil (9.149).

H.p.l.c. Rₜ=2.76 min.

¹H nmr (CDCl₃) δ: 1.88 (1H, t), 2.75 (2H, t), 3.86 (2H, q), 7.01 (1H, m), 7.10 (1H, m), 7.17 (1H, m).

### Intermediate 26

### 4-(2,3-Difluorophenyl)-1-butanol

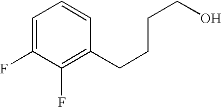

A solution of 4-(2,3-Difluorophenyl)-3-butyn-1-ol (see Intermediate 25) (9.12 g, 50.06 mmol) in ethanol (200 ml) was stirred under hydrogen (50 p.s.i) at ambient temperature over palladium hydroxide on carbon (2.0 g, 20% palladium by weight, E101 NE/W) for 20 h. The catalyst was then removed by filtration and the solution evaporated under reduced pressure, washed with EtOH (200 ml) and the filtrate evaporated to dryness to afford the title compound (8.88 G) as a grey oil.

H.p.l.c. Rₜ=2.87 min.

¹H nmr (CDCl₃) δ: 1.38 (br OH under H₂O peak), 1.64 (2H, m), 1.71 (2H, m), 2.71 (2H, dt), 3.69 (2H, t), 6.92-7.03 (3H, m).

### Intermediate 27

### 4-(2,3-Difluorophenyl)butanoic acid

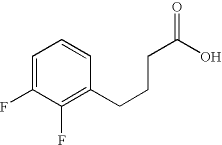

To a solution of 4-(2,3-Difluorophenyl)-1-butanol (see Intermediate 26) (8.87 g) in dry dimethyl formamide (86 ml) cooled to −2° C. under nitrogen was added pyridinium dichromate (45.8 g) in portions over 20 minutes. Cooling was removed after 1 h and the mixture stirred for 72 h at ambient temperature, after which 400 ml of water was added and the mixture vigorously stirred and extracted with 5×150 ml of ether. The combined ether layers were washed with brine, separated and dried by passage though a hydrophobic frit, then evaporated. The crude mixture was combined with material from a similar reaction (performed on 2.68 g scale) and purified on 2×70 g amino propyl solid phase extraction cartridges, loaded and washed with methanol, then eluted with 5% 5N aqueous HCl in methanol, affording 4.31 g of a yellow oil, a mixture of the title compound and its methyl ester. This mixture was dissolved in 100 ml of 1:1 methanol water, to which was added 20 ml of 2N aqueous sodium hydroxide. After stirring at room temperature for 18 h the majority of volatiles were removed and the remaining aqueous residue acidified with 2N HCl and extracted with 3×50 ml of dichloromethane. The combined organic fractions were washed with brine, dried through a hydrophobic frit and evaporated to dryness to yield the title compound (4.05 g) as a pale pink waxy solid.

Mass spectrum: Found: [M-H]-199

H.p.l.c. Rₜ=2.89 min.

### Intermediate 28

### 5,6-Difluoro-3,4-dihydro-1(2H)-naphthalenone

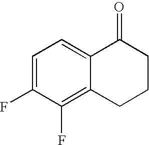

A mixture of methanesulphonic acid (85 ml) and phosphorus pentoxide (8.5 g) was stirred for 18 h at room temperature under nitrogen with Eaton's reagent for 3 h followed by under a drying tube with silica gel for 18 h. 4-(2,3-Difluorophenyl)butanoic acid (4.0 g, 19.98 mmol) was added to this in four portions over 10 minutes and the mixture vigorously stirred for 6.5 h, when it was added slowly to 300 ml of water kept <−25° C. with external ice cooling, then stirred from an additional 20 min. This mixture was extracted with 3×100 ml portions of dichloromethane, which were combined, washed with brine, dried through a hydrophobic frit and evaporated to dryness, affording the title compound (3.57 g) as a light orange oil

H.p.l.c. Rₜ=2.90 min.

¹H nmr (CDCl₃) δ: 2.17 (2H, m), 2.66 (2H, t), 3.00 (2H, t), 7.12 (1H, m), 7.86 (1H, m).

### Intermediate 29

### 6-Amino-5-fluoro-3,4-dihydro-1(2H)-naphthalenone

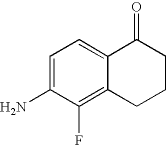

A solution of 5,6-Difluoro-3,4-dihydro-1(2H)-naphthalenone (3.03 g, 16.6 mmol) in 2M ammonia in methanol was stirred with 60 ml of aqueous ammonia (density 0.88 g/l) in an autoclave at 160° C., 300 psi for 17 h. The mixture was cooled to ambient temperature and the mixture was filtered, well-washed with water/methanol and dried in vacuo at 60° C. overnight, affording the title compound (1.77 g) as brown needles.

Mass spectrum: Found: MH⁺ 180

H.p.l.c. Rₜ 2.39 min

### Intermediate 30

### 6-Amino-5-fluoro-3,4-dihydro-1(2H)-naphthalenone oxime

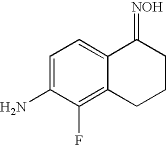

Prepared from 6-amino-5-fluoro-3,4-dihydro-1(2H)-naphthalenone (420 mg, 2.34 mmol) using a procedure similar to that described for Intermediate 1

Mass spectrum: Found: MH⁺ 195

H.p.l.c. Rₜ 2.52 min

### Intermediate 31

### 7-amino-6-fluoro-2,3,4,5-tetrahydro-1H-2-benzazepin-1-one

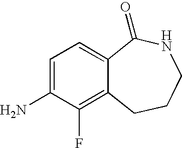

Prepared from 6-Amino-5-fluoro-3,4-dihydro-1(2H)-naphthalenone oxime (410 mg) using a procedure similar to that described for Intermediate 2

Mass spectrum: Found: MH⁺ 195

H.p.l.c. Rₜ 1.92 min

### Intermediate 32

### 6-Fluoro-2,3,4,5-tetrahydro-1H-2-benzazepin-7-amine

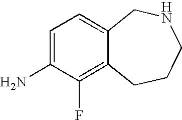

Prepared from 7-amino-6-fluoro-2,3,4,5-tetrahydro-1H-2-benzazepin-1-one (190 mg) using a procedure similar to that described for Intermediate 3

Mass spectrum: Found: MH⁺ 181

H.p.l.c. Rₜ 0.58 min

### Intermediate 33

### 1,1-Dimethylethyl 7-amino-6-fluoro-1,3,4,5-tetrahydro-2H-2-benzazepine-2-carboxylate

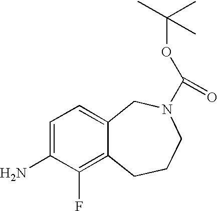

Prepared from 6-Fluoro-2,3,4,5-tetrahydro-1H-2-benzazepin-7-amine (1.076 mmol) using a procedure similar to that described for Intermediate 4, utilising dichloromethane (100 ml) in lieu of dioxane.

Mass spectrum: Found: MH⁺ 281.

H.p.l.c. Rₜ=3.06 min.

### Intermediate 34

### 1,1-Dimethylethyl 6-fluoro-7-[(N-{[(Phenylmethyl)oxy]carbonyl}-L-methionyl)amino]-1,3,4,5-tetrahydro-2H-2-benzazepine-2-carboxylate

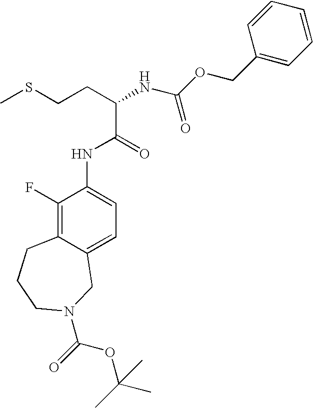

Prepared from 1,1-Dimethylethyl 7-amino-6-fluoro-1,3,4,5-tetrahydro-2H-2-benzazepine-2-carboxylate (106 mg, 0.378 mmol) using a procedure similar to that described for Intermediate 5.

Mass spectrum: Found: MH⁺ 546.

H.p.l.c. Rₜ=3.58 min.

### Intermediate 35

### [(3S)-4-[(2-{[(1,1-Dimethylethyl)oxy]-carbonyl}-6-fluoro-2,3,4,5-tetrahydro-1H-2-benzazepin-7-yl)amino]-4-oxo-3-({[(phenylmethyl)oxy]carbonyl}amino)butyl](dimethyl)sulfonium iodide

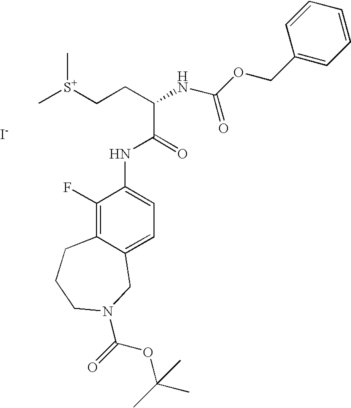

Prepared from [(3S)-4-[(2-{[(1,1-Dimethylethyl)oxy]carbonyl}-6-fluoro-2,3,4,5-tetrahydro-1H-2-benzazepin-7-yl)amino]-4-oxo-3-({[(phenylmethyl)oxy]carbonyl}amino)butyl](dimethyl)sulfonium iodide using the a procedures similar to that described for Intermediate 6.

Mass spectrum: Found: M⁺ 560

H.p.l.c. Rₜ=2.67 min.

### Intermediate 36

### 1,1-Dimethylethyl 6-fluoro-7-[(3S)-2-oxo-3-({[(Phenylmethyl)oxy]carbonyl}amino)-1-pyrrolidinyl]-1,3,4,5-tetrahydro-2H-2-benzazepine-2-carboxylate

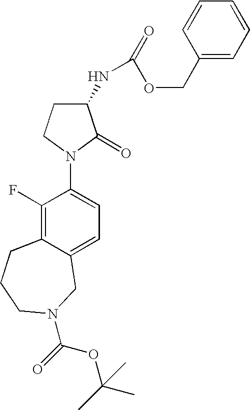

Prepared from 1,1-Dimethylethyl 6-fluoro-7-[(N-{[(phenylmethyl)oxy]carbonyl}-L-methionyl)amino]-1,3,4,5-tetrahydro-2H-2-benzazepine-2-carboxylate (78 mg) using the a procedure similar to that described for Intermediate 7.

Mass spectrum: Found: MH⁺ 498

H.p.l.c. Rₜ=3.34 min.

### Intermediate 37

### 1,1-Dimethylethyl 7-[(3S)-3-amino-2-oxo-1-pyrrolidinyl]-6-fluoro-1,3,4,5-tetrahydro-2H-2-benzazepine-2-carboxylate

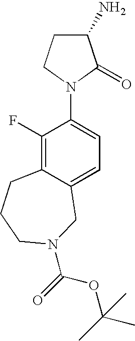

Prepared from 1,1-Dimethylethyl 6-fluoro-7-[(3S)-2-oxo-3-({[(phenylmethyl)oxy]carbonyl}amino)-1-pyrrolidinyl]-1,3,4,5-tetrahydro-2H-2-benzazepine-2-carboxylate using a procedure similar to that described for Intermediate 8.

Mass spectrum: Found: MH⁺ 364

H.p.l.c. Rₜ=2.25 min.

### Intermediate 38

### 1,1-Dimethylethyl 7-[(3S)-3-({[(E)-2-(5-chloro-2-thienyl)ethenyl]sulfonyl}amino)-2-oxo-1-pyrrolidinyl]-6-fluoro-1,3,4,5-tetrahydro-2H-2-benzazepine-2-carboxylate

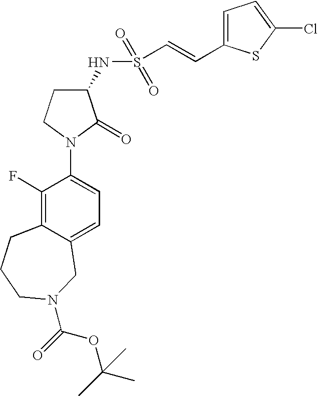

Prepared from 1,1-Dimethylethyl 7-[(3S)-3-amino-2-oxo-1-pyrrolidinyl]-6-fluoro-1,3,4,5-tetrahydro-2H-2-benzazepine-2-carboxylate (30 mg, 0.083 mmol) and (E)-2-(5-chloro-2-thienyl)ethanesulfonyl chloride (21.2 mg, 0.087 mmol) using a procedure similar to that described for Intermediate 9.

Mass spectrum: Found: MH⁺ 570, 572

H.p.l.c. Rₜ=3.55 min.

### Intermediate 39

### 1,1-Dimethylethyl 7-((3S)-3-{[(6-chloro-2-naphthalenyl)sulfonyl]-amino}-2-oxo-1-pyrrolidinyl)-6-fluoro-1,3,4,5-tetrahydro-2H-2-benzazepine-2-carboxylate

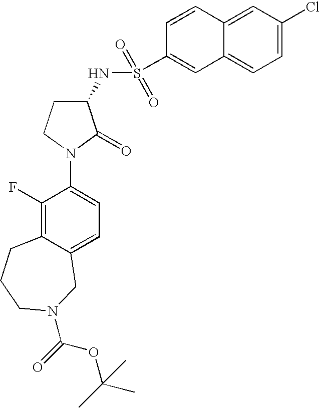

Prepared from 1,1-Dimethylethyl 7-[(3S)-3-amino-2-oxo-1-pyrrolidinyl]-6-fluoro-1,3,4,5-tetrahydro-2H-2-benzazepine-2-carboxylate (30 mg, 0.083 mmol) and 6-chloro-2-naphthalenesulfonyl chloride (23 mg, 0.087 mmol) using a procedure similar to that described for Intermediate 9.

Mass spectrum: Found: MH⁺ 588, 590

H.p.l.c. Rₜ=3.68 min.

### Example 1a

### (E)-2-(5-Chloro-2-thienyl)-N-[(3S)-2-oxo-1-(2,3,4,5-tetrahydro-1H-2-benzazepin-7-yl)-3-pyrrolidinyl]ethenesulfonamide hydrochloride (form 1)

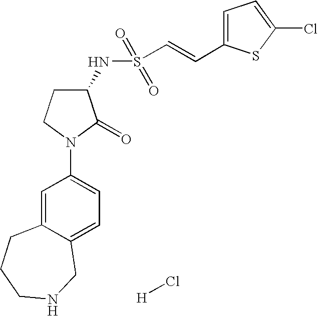

1,1-Dimethylethyl 7-[(3S)-3-({[(E)-2-(5-chloro-2-thienyl)ethenyl]sulfonyl}amino)-2-oxo-1-pyrrolidinyl]-1,3,4,5-tetrahydro-2H-2-benzazepine-2-carboxylate (see Intermediate 9) (12.40 g, 22.5 mmol) was stirred in 4M HCl in dioxane (300 ml) in a stoppered flask covered in foil for 4 days at room temperature. The reaction mixture was then evaporated under reduced pressure to afford a yellow solid. The residue was triturated with diethyl ether (500 ml) and the yellow solid collected by filtration, washed with diethyl ether (750 ml) and dried in vacuo at room temperature for 1 h/2 h. This material was heated to reflux in EtOH (3000 ml) and water (60 ml) and filtered while hot. Approximately 1000 ml of EtOH was then removed via distillation and the remaining solution (some evidence of product crystallisation at this point) was allowed to cool to room temperature overnight. The crystallised solid was then removed by filtration. Half the material was refluxed with a distillation set-up to remove solvent (approximately 500 ml). The resulting cloudy mixture was cooled slightly and the rest of the mixture was added. The slight suspension was heated to reflux and 500 ml solvent was distilled off. The suspension was left in a coiling oil-bath for 2 h, removed from the oil bath (still quite warm), covered in foil and allowed to cool to room temperature overnight. The flocculent precipitate was collected by filtration, washed with EtOH and dried in vacuo at 60° C. for 20 h to afford the title compound (7.34 g) as an off-white solid. A second crop (1.46 g) was isolated through further concentration of the mother liquor.

Mass spectrum: Found: MH⁺ 452, 454

H.p.l.c. Rₜ=2.42 min.

¹H NMR (d₆-DMSO) δ: 1.90 (3H, m), 2.47 (1H, m), 2.96 (2H, m), 3.31 (2H, m), 3.73 (2H, m), 4.27 (3H, m), 6.98 (1H, d), 7.20 (1H, d), 7.40-7.54 (5H, m), 8.03 (1H, d), 9.06 (2H, br. s).

### Example 1b

### (E)-2-(5-Chloro-2-thienyl)-N-[(3S)-2-oxo-1-(2,3,4,5-tetrahydro-1H-2-benzazepin-7-yl)-3-pyrrolidinyl]ethenesulfonamide hydrochloride hydrate

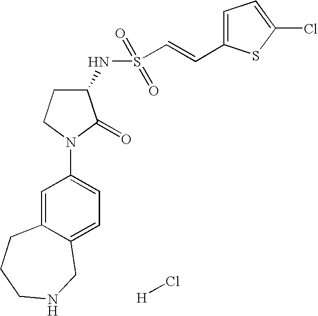

1,1-Dimethylethyl 7-[(3S)-3-({[(E)-2-(5-chloro-2-thienyl)ethenyl]sulfonyl}amino)-2-oxo-1-pyrrolidinyl]-1,3,4,5-tetrahydro-2H-2-benzazepine-2-carboxylate (see Intermediate 9) (18.24 g, 33.0 mmol) was stirred in 4M HCl in dioxane (460 ml) in a stoppered flask covered in foil for 65 h at room temperature. The reaction mixture was then evaporated under reduced pressure to afford a yellow solid. This solid was suspended in diethyl ether (600 ml) and stirred at room temperature for 2.5 h before it was collected by filtration, washed with more diethyl ether (600 ml), sucked dry and dried in vacuo at 50° C. to give a pale cream solid. This material was then suspended in EtOH (250 ml) and stirred at room temperature for 1 h. The solid was stirred in EtOH at room temperature then collected via filtration, washed in EtOH and dried in a series of stages in vacuo at room temperature to 50° C. for a total of approximately 37-42 h (i.e. room temperature for 2 h, room temperature for 1.5 h, room temperature for 16.5 h, in vacuo at 50° C. for 1 h 45 mins, in vacuo at 50° C. for approximately 2 h and in vacuo at 45° C. for 15 h 45 mins) to afford the title compound (15.75 g) as a pale cream solid.

Mass spectrum: Found: MH⁺ 452, 454

H.p.l.c. Rₜ=2.42 min.

¹H NMR (d₆-DMSO) δ: 1.90 (3H, m), 2.47 (1H, m), 2.96 (2H, m), 3.31 (2H, m), 3.73 (2H, m), 4.27 (3H, m), 6.98 (1H, d), 7.20 (1H, d), 7.40-7.54 (5H, m), 8.03 (1H, d), 9.13 (2H, br. s).

### Example 1c

### (E)-2-(5-Chloro-2-thienyl)-N-[(3S)-2-oxo-1-(2,3,4,5-tetrahydro-1H-2-benzazepin-7-yl)-3-pyrrolidinyl]ethenesulfonamide

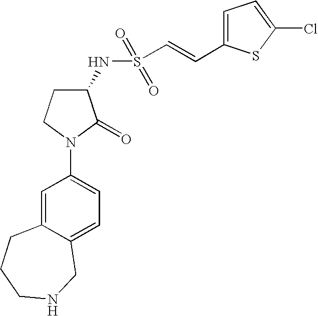

(E)-2-(5-Chloro-2-thienyl)-N-[(3S)-2-oxo-1-(2,3,4,5-tetrahydro-1H-2-benzazepin-7-yl)-3-pyrrolidinyl]ethenesulfonamide hydrochloride (see Example 1a) (5.58 g, 11.42 mmol; form 1) was stirred in saturated aqueous sodium carbonate solution (100 ml) for 5 min before DCM (500 ml) was added. The mixture was stirred vigorously for 65 min, then diluted with water (20 ml), shaken vigorously and the layers were separated. The aqueous phase was extracted with DCM (50 ml) and the combined organic phases were passed through a hydrophobic frit and evaporated under reduced pressure and dried in vacuo at room temperature to leave the title compound as a yellow-orange solid (5.25 g).

Mass spectrum: Found: MH⁺ 452, 454

H.p.l.c. Rₜ=2.38 min.

¹H NMR (d₆-DMSO) δ: 1.58 (2H, m), 1.91 (1H, m), 2.47 (1H, m), 2.85 (2H, m), 3.01 (2H, m), 3.34 (exchangeable protons and HOD, br.s), 3.74 (2H, m), 3.72 (2H, m), 4.22 (1H, dd), 6.98 (1H, d), 7.10 (1H, d), 7.20 (1H, d), 7.35 (1H, dd), 7.39 (1H, m), 7.43 (1H, d), 7.50 (1H, d).

### Example 1d

### (E)-2-(5-Chloro-2-thienyl)-N-[(3S)-2-oxo-1-(2,3,4,5-tetrahydro-1H-2-benzazepin-7-yl)-3-pyrrolidinyl]ethenesulfonamide hydrochloride (form 2)

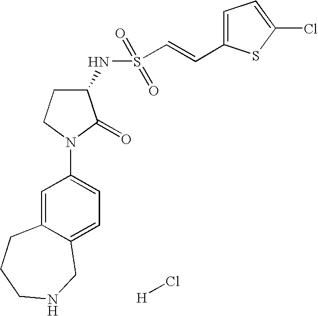

(E)-2-(5-Chloro-2-thienyl)-N-[(3S)-2-oxo-1-(2,3,4,5-tetrahydro-1H-2-benzazepin-7-yl)-3-pyrrolidinyl]ethenesulfonamide (see Example 1c) (0.101 g, 022 mmol) was stirred and sonicated in dry EtOH (2 ml) for approximately 5 mins, then more dry EtOH (2 ml) was added and the mixture was warmed to 45° C. under nitrogen and then stirred at room temperature. 2M HCl in diethyl ether (0.4 ml) was added and the mixture was stirred for 90 min before the solid was collected by filtration, then washed with a small amount of EtOH and dried in vacuo at room temperature to give the title compound (0.093 g) as a white solid.

Mass spectrum MH⁺, H.p.l.c. Rₜ and ¹H NMR (d₆-DMSO) δ values concordant with those described for Examples 1a and 1b.

### Example 1e

### (E)-2-(5-Chloro-2-thienyl)-N-[(3S)-2-oxo-1-(2,3,4,5-tetrahydro-1H-2-benzazepin-7-yl)-3-pyrrolidinyl]ethenesulfonamide hydrochloride (form 1)

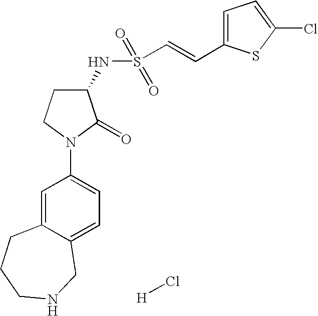

A 1:1 mixture of (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride (see Example 1a) and (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride (Example 1d) (0.08 g) suspended in EtOH (6 ml) and then partitioned into 2 tubes was slurried at room temperature for 7 days, one at room temperature and the other at 50° C. The mixture was filtered and the solid was collected by filtration, then washed with diethyl ether and dried in vacuo to provide the title compound as a white solid (XRPD concordant with Example 1a).

### Example 1f

### (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hemisuccinate

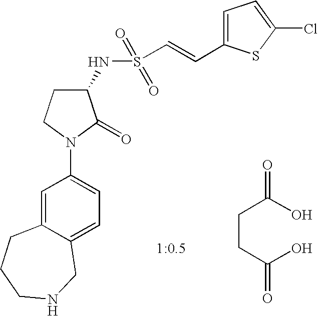

MeOH (3 ml) was added to the free base of (E)-2-(5-Chloro-2-thienyl)-N-[(3S)-2-oxo-1-(2,3,4,5-tetrahydro-1H-2-benzazepin-7-yl)-3-pyrrolidinyl]ethenesulfonamide (see Example 1c) (350 mg, 77.4 mmol) and this was heated at 60° C. for an hour to dissolve the free base. To succinic acid (96 mg, 1.05 eq), MeOH (500 μl) was added to dissolve the acid. The acid solution was added to the freebase solution and this was left to temperature cycle (0°-40° C.) for 2 days. The white solid formed was isolated, washed with MeOH. The solid was left on the filter paper for a couple of hours and then in the vacuum oven at 40° C. overnight to provide the title compound as a white solid (355.6 mg).

Mass spectrum: Found: MH⁺ 452, 454

¹H NMR (d₆-DMSO) δ: 1.65 (2H, m), 1.90 (1H, q), 2.25 (2H, s), 2.45 (m, 1H), 2.90 (2H, d), 3.10 (2H, m), 3.70 (2H, m), 3.92 (2H, s), 4.22 (1H, m), 6.95 (1H, d), 7.18 (2H, m), 7.42 (4H, m)

### Thermal Analysis

The DSC thermogram were obtained using a TA Q1000 calorimeter, serial number 1000-0126. The samples were weighed into an aluminium pan, a pan lid placed on top and lightly crimped without sealing the pan. The experiment was conducted using a heating rate of 10° C. min⁻¹.

Melting onset for substantially crystalline forms 1 and 2 of (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride were in the range 264-276° C.

Melting onset for substantially crystalline (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hemisuccinate was in the range 222-224° C.

### X-Ray Powder Diffraction (XRPD)

X-ray powder diffraction (XRPD) data are shown in FIGS. 1-5. The data were acquired on a PANalytical X'Pert Pro powder diffractometer, model PW3040/60, serial number DY1850 using an X'Celerator detector. The acquisition conditions were: radiation: Cu Kα, generator tension: 40 kV, generator current: 45 mA, start angle: 2.0°2θ, end angle: 40.0°2 θ, step size: 0.0167 °2 θ, time per step: 31.75 seconds. The samples were prepared by mounting a few milligrams of sample on a Si wafer (zero background) plates, resulting in a thin layer of powder. Characteristic XRPD angles and d-spacings are recorded in Tables 1 and 2.

Note that for substantially crystalline (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride form 2, a peak at approximately 16.5° 2θ has been omitted from Table 1 as it was not seen for a previous batch of this form, hence it may be due to a minor impurity (chemical or phase).

Characteristic peaks for this solid state form are summarised in Table 1 with 2θ and calculated lattice spacings. Peak positions and corresponding d-spacings were calculated using Highscore software.

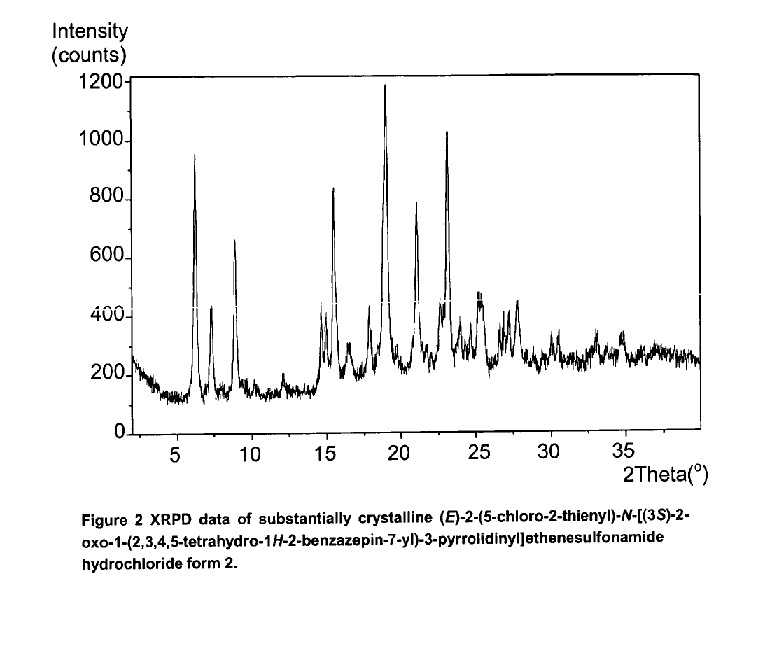

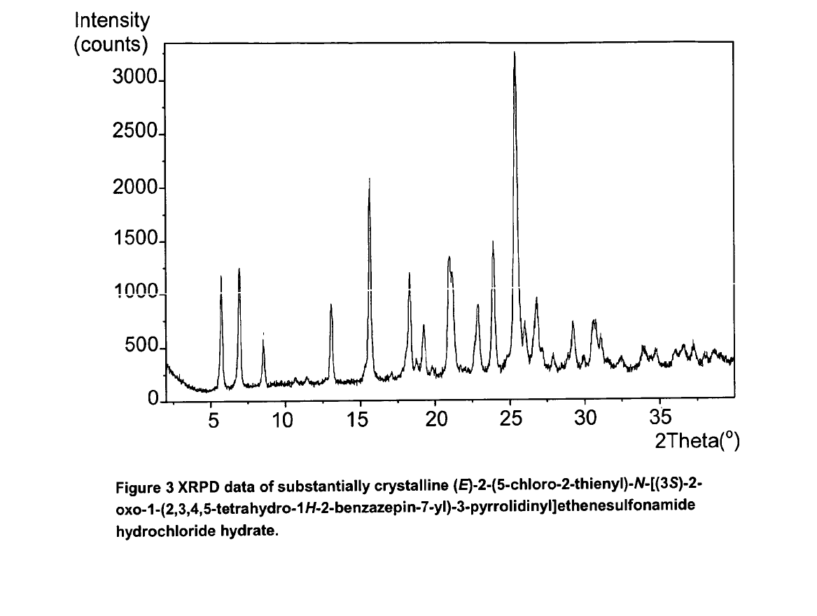

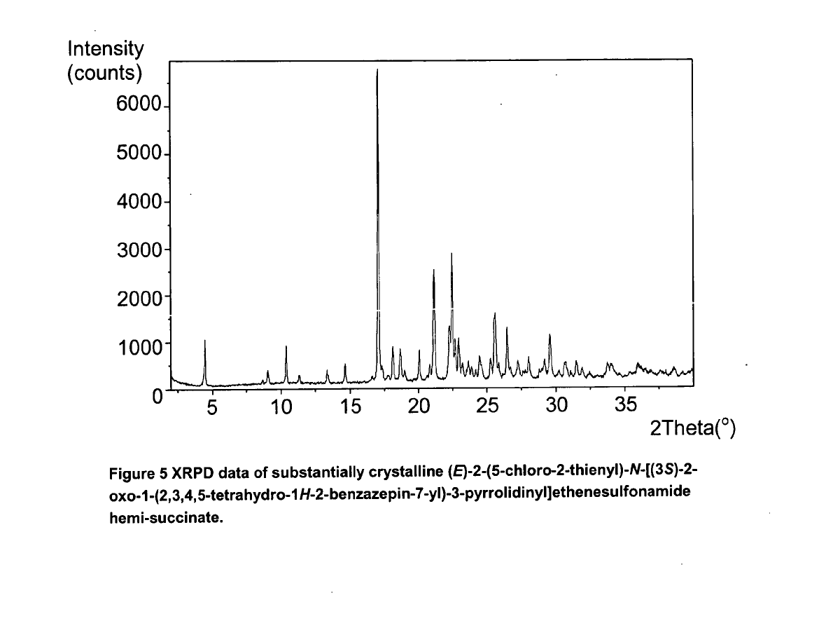

FIG. 1 shows XRPD data of substantially crystalline (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride form 1. FIG. 2 shows XRPD data of substantially crystalline (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride form 2. FIG. 3 shows XRPD data of substantially crystalline (E)-2-(5-chloro-2-thienyl)-N-[(3S)-2-oxo-1-(2,3,4,5-tetrahydro-1H-2 benzazepin-7-yl)-3-pyrrolidinyl]ethenesulfonamide hydrochloride hydrate. FIG. 4 shows an overlay of XRPD data for Form 1 (bottom), Form 2 (middle) and Hydrate (top). The data are offset for clarity. FIG. 5 shows XRPD data of substantially crystalline (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hemi-succinate.

TABLE 1
XRPD peak data for substantially crystalline (E)-2-(5-chloro-2-
thienyl)-N-[(3S)-2-oxo-1-(2,3,4,5-tetrahydro-1H-2-benzazepin-7-
yl)-3-pyrrolidinyl]ethenesulfonamide hydrochloride form 1 (Example
1a), substantially crystalline (E)-2-(5-chloro-2-thienyl)-N-[(3S)-
2-oxo-1-(2,3,4,5-tetrahydro-1H-2-benzazepin-7-yl)-3-
pyrrolidinyl]ethenesulfonamide hydrochloride form 2 (Example 1d)
and substantially crystalline (E)-2-(5-chloro-2-thienyl)-N-[(3S)-
2-oxo-1-(2,3,4,5-tetrahydro-1H-2-benzazepin-7-yl)-3-
pyrrolidinyl]ethenesulfonamide hydrochloride hydrate (Example 1b).

| Example 1a: Form 1 | | Example 1d: Form 2 | | Example 1b: Hydrate | |
|---|---|---|---|---|---|
| 2θ/° | d-spacing/Å | 2θ/° | d-spacing/Å | 2θ/° | d-spacing/Å |
| 6.2 | 14.1 | 6.3 | 14.1 | 5.7 | 15.4 |
| 8.0 | 11.0 | 7.4 | 12.0 | 7.0 | 12.7 |
| 9.5 | 9.3 | 8.9 | 9.9 | 8.5 | 10.4 |
| 12.5 | 7.1 | 10.2 | 8.7 | 13.0 | 6.8 |
| 14.9 | 6.0 | 12.1 | 7.3 | 15.6 | 5.7 |
| 16.4 | 5.4 | 14.7 | 6.0 | 18.3 | 4.8 |
| 18.7 | 4.7 | 15.0 | 5.9 | 19.2 | 4.6 |
| 19.1 | 4.6 | 15.5 | 5.7 | 20.9 | 4.2 |
| 19.6 | 4.5 | 17.9 | 5.0 | 21.2 | 4.2 |
| 20.8 | 4.3 | 19.0 | 4.7 | 22.9 | 3.9 |
| 21.3 | 4.2 | 21.1 | 4.2 | 23.9 | 3.7 |
| 23.6 | 3.8 | 22.6 | 3.9 | 25.3 | 3.5 |
| 23.8 | 3.7 | 23.1 | 3.8 | 26.0 | 3.4 |
| 25.3 | 3.5 | 24.0 | 3.7 | 26.8 | 3.3 |
| 30.0 | 3.0 | 24.7 | 3.6 | 29.2 | 3.1 |
| | | 25.2 | 3.5 | | |
| | | 26.6 | 3.4 | | |
| | | 26.9 | 3.3 | | |
| | | 27.2 | 3.3 | | |
| | | 27.8 | 3.2 | | |

TABLE 2
XRPD data for substantially crystalline (E)-2-(5-chloro-2-thienyl)-
N-[(3S)-2-oxo-1-(2,3,4,5-tetrahydro-1H-2-benzazepin-7-yl)-3-
pyrrolidinyl]ethenesulfonamide hemisuccinate (Example 1f)

| 2θ/° | d-spacing/Å |
|---|---|
| 4.5 | 19.7 |
| 8.7 | 10.2 |
| 9.1 | 9.8 |
| 10.4 | 8.5 |
| 11.3 | 7.8 |
| 13.3 | 6.6 |
| 14.6 | 6.1 |
| 17.1 | 5.2 |
| 18.1 | 4.9 |
| 18.7 | 4.8 |
| 20.0 | 4.4 |
| 21.1 | 4.2 |
| 22.5 | 4.0 |
| 22.9 | 3.9 |
| 24.5 | 3.6 |
| 25.5 | 3.5 |
| 26.4 | 3.4 |
| 27.2 | 3.3 |
| 28.0 | 3.2 |
| 29.6 | 3.0 |

### Example 2

### 6-Chloro-N-[(3S)-2-oxo-1-(2,3,4,5-tetrahydro-1H-2-benzazepin-7-yl)-3-pyrrolidinyl]-2-naphthalenesulfonamide hydrochloride

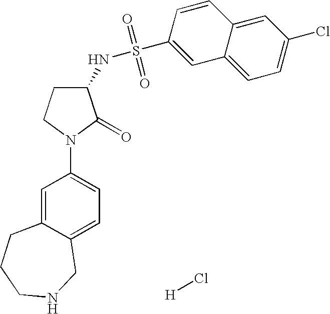

Prepared from 1,1-Dimethylethyl 7-[(3S)-3-amino-2-oxo-1-pyrrolidinyl]-1,3,4,5-tetrahydro-2H-2-benzazepine-2-carboxylate (see Intermediate 8) and 6-chloro-2-naphthalenesulfonyl chloride using a procedure similar to that described for Intermediate 9 followed by a deprotection procedure similar to that described for Example 1b.

Mass spectrum: Found: MH⁺ 470, 472

H.p.l.c. Rₜ=2.58 min

¹H NMR (d₆-DMSO) δ: 1.68-1.82 (3H, m), 2.15 (1H, m), 2.93 (2H, m), 3.30 2H, m), 3.63 (2H, m), 4.25 (2H, s), 4.34 (1H, m), 7.37 (1H, d), 7.45 (2H, m), 7.69 (1H, dd), 7.95 (1H, dd), 8.13 (1H, d), 8.23 (2H, m), 8.45 (1H, m), 8.55 (1H, m), 8.95 (2H, br. s).

### Example 3

### 3-Chloro-N-[(3S)-2-oxo-1-(2,3,4,5-tetrahydro-1H-2-benzazepin-7-yl)-3-pyrrolidinyl]-1H-indole-6-sulfonamide hydrochloride

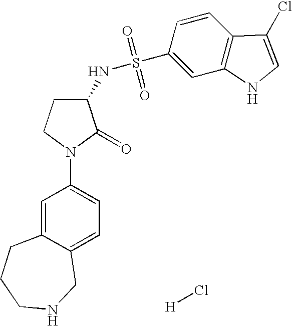

Prepared from 1,1-dimethylethyl 7-((3S)-3-{[(3-chloro-1H-indol-6-yl)sulfonyl]amino}-2-oxo-1-pyrrolidinyl)-1,3,4,5-tetrahydro-2H-2-benzazepine-2-carboxylate (see Intermediate 12) using a procedure similar to that described for Example 1b.

Mass spectrum: Found: MH⁺ 457, 459

H.p.l.c. Rₜ=2.42 min

¹H NMR (d₆-DMSO) δ: 1.66 (1H, m), 1.82 (2H, m), 2.04 (1H, m), 2.93 (2H, m), 3.31 (2H, m), 3.61 (2H, m), 4.23 (3H, m), 7.37 (1H, d), 7.47 (2H, m), 7.60 (1H, dd), 7.67 (1H, d), 7.83 (1H, d), 7.98 (1H, s), 8.17 (1H, d), 8.86 (2H, br. s), 11.94 (1H, s).

### Example 4

### 6-Chloro-N-[(3S)-1-(2-methyl-2,3,4,5-tetrahydro-1H-2-benzazepin-7-yl)-2-oxo-3-pyrrolidinyl]-2-naphthalenesulfonamide formate

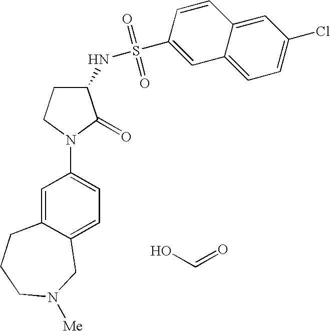

6-Chloro-N-[(3S)-2-oxo-1-(2,3,4,5-tetrahydro-1H-2-benzazepin-7-yl)-3-pyrrolidinyl]-2-naphthalenesulfonamide hydrochloride (see Example 2) (125 mg, 0.247 mmol) was suspended in chloroform (10 ml). Paraformaldehyde (37 mg, 1.23 mmol) and formic acid (47 ul) were added and the mixture was heated at reflux for 1.5 h. On cooling, the solution was evaporated under reduced pressure and remainder made up in 2 ml 1:1 DMSO:MeOH for autoprep. Mixture was purified using mass directed preparative h.p.l.c. followed by trituration with diethyl ether to afford the title compound (20.5 mg) as an off-white solid.

Mass spectrum: Found: MH⁺ 484, 486

H.p.l.c. Rₜ=2.59 min

¹H NMR (d₄-MeOD) δ: 1.93 (3H, m), 2.34 (1H, m), 2.79 (3H, s), 2.95 (2H, s), 3.49 (2H, m), 3.71 (2H, m), 4.31 (1H, dd), 4.40 (2H, br. s), 7.32 (1H, d), 7.44 (1H, dd), 7.50 (1H, d), 7.56 (1H, dd), 7.99 (4H, m), 8.41 (1H, s), 8.51 (1H, s)

### Example 5

### (E)-2-(5-Chloro-2-thienyl)-N-[(3S)-2-oxo-1-(2,3,4,5-tetrahydro-1H-3-benzazepin-7-yl)-3-pyrrolidinyl]ethenesulfonamide hydrochloride

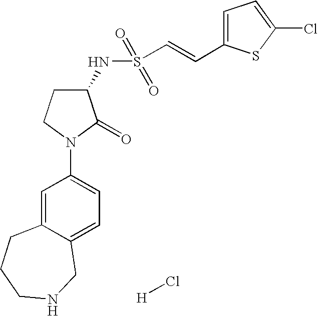

Prepared from 1,1-dimethylethyl 7-[(3S)-3-amino-2-oxo-1-pyrrolidinyl]-1,2,4,5-tetrahydro-3H-3-benzazepine-3-carboxylate (see Intermediate 13) and (E)-2-(5-chloro-2-thienyl)ethanesulfonyl chloride using a procedure similar to that described for Intermediate 9 followed by a deprotection procedure similar to that described for Example 1b.

Mass spectrum: Found: MH⁺ 452, 454

H.p.l.c. Rₜ=2.73 min

### Example 6

### 6-Chloro-N-[(3S)-2-oxo-1-(2,3,4,5-tetrahydro-1H-3-benzazepin-7-yl)-3-pyrrolidinyl]-2-naphthalenesulfonamide hydrochloride

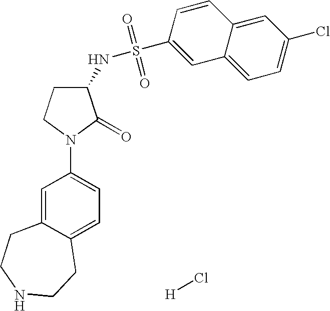

Prepared from 1,1-dimethylethyl 7-[(3S)-3-amino-2-oxo-1-pyrrolidinyl]-1,2,4,5-tetrahydro-3H-3-benzazepine-3-carboxylate (see Intermediate 13) and 6-chloro-2-naphthalenesulfonyl chloride using a procedure similar to that described for Intermediate 9 followed by a deprotection procedure similar to that described for Example 1b.

Mass spectrum: Found: MH⁺ 470, 472

H.p.l.c. Rₜ=2.82

### Example 7

### 3-Chloro-N-[(3S)-2-oxo-1-(2,3,4,5-tetrahydro-1H-3-benzazepin-7-yl)-3-pyrrolidinyl]-1H-indole-6-sulfonamide hydrochloride

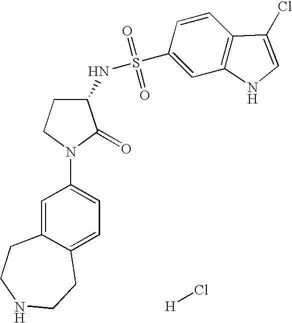

Prepared from 1,1-dimethylethyl 7-((3S)-3-{[(3-chloro-1H-indol-6-yl)sulfonyl]amino}-2-oxo-1-pyrrolidinyl)-1,2,4,5-tetrahydro-3H-3-benzazepine-3-carboxylate

(see Intermediate 14) using a procedure similar to that described for Example 1b.

Mass spectrum: Found: MH⁺ 459, 461

H.p.l.c. Rₜ=2.72

### Example 8

### (E)-2-(5-Chloro-2-thienyl)-N-[(3S)-2-oxo-1-(2,3,4,5-tetrahydro-1H-2-benzazepin-8-yl)-3-pyrrolidinyl]ethenesulfonamide hydrochloride

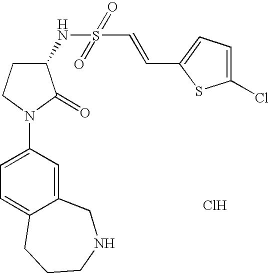

A solution of 1,1-dimethylethyl 8-[(3S)-3-({[(E)-2-(5-chloro-2-thienyl)ethenyl]sulfonyl}amino)-2-oxo-1-pyrrolidinyl]-1,3,4,5-tetrahydro-2H-2-benzazepine-2-carboxylate (see Intermediate 18) (0.732 g) in EtOH (10 ml) was treated with 2M HCl in diethyl ether (50 ml). After stirring at room temperature for 64 h the resulting solid was collected by filtration, washed with diethyl ether and dried in vacuo at 45° C. to give the title compound (0.606 g) as a cream solid.

Mass spectrum: Found: MH⁺ 452, 454

H.p.l.c. Rₜ=2.43 min.

¹H NMR (d₆-DMSO) δ: 1.83 (2H, m), 1.95 (1H, m), 2.50 (1H, m, partially obscured by DMSO-d5 signal), 2.95 (2H, m), 3.32 (2H, m), 3.73 (2H, m), 4.27 (3H, m), 6.99 (1H, d), 7.20 (1H, d), 7.27 (1H, d), 7.43 (1H, d), 7.50 (1H, d), 7.61 (1H, d), 7.66 (1H, s), 8.04 (1H, d), 9.27 (2H, br. s).

### Example 9

### 6-Chloro-N-[(3S)-2-oxo-1-(2,3,4,5-tetrahydro-1H-2-benzazepin-8-yl)-3-pyrrolidinyl]-2-naphthalenesulfonamide hydrochloride

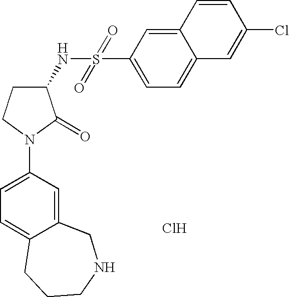

Prepared from 1,1-dimethylethyl 8-[(3S)-3-amino-2-oxo-1-pyrrolidinyl]-1,3,4,5-tetrahydro-2H-2-benzazepine-2-carboxylate (see Intermediate 17) and 6-chloro-2-naphthalenesulfonyl chloride using a procedure similar to that described for Intermediate 9 followed by a deprotection procedure similar to that described for Example 8.

Mass spectrum: Found: MH⁺ 470, 472

H.p.l.c. Rₜ=2.59 min.

¹H NMR (d₆-DMSO) δ: 1.75 (1H, m), 1.81 (2H, m), 2.17 (1H, m), 2.92 (2H, m), 3.29 (2H, m), 3.63 (2H, m), 4.26 (2H, s), 4.33 (1H, m), 7.23 (1H, d), 7.52 (1H, d), 7.60 (1H, s), 7.69 (1H, d), 7.97 (1H, d), 8.13 (1H, d), 8.21 (2H, m), 8.48 (1H, d), 8.55 (1H, s), 9.21 (2H, br. s).

### Example 10

### (E)-2-(4-Chlorophenyl)-N-[(3S)-2-oxo-1-(2,3,4,5-tetrahydro-1H-2-benzazepin-8-yl)-3-pyrrolidinyl]ethenesulfonamide hydrochloride

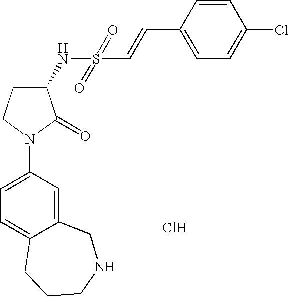

Prepared from 1,1-dimethylethyl 8-[(3S)-3-amino-2-oxo-1-pyrrolidinyl]-1,3,4,5-tetrahydro-2H-2-benzazepine-2-carboxylate (see Intermediate 17) and (E)-2-(4-chlorophenyl)ethanesulfonyl chloride using a procedure similar to that described for Intermediate 9 followed by a deprotection procedure similar to that described for Example 8.

Mass spectrum: Found: MH⁺ 446, 448

H.p.l.c. Rₜ=2.48 min.

¹H NMR (d₆-DMSO) δ: 1.83 (2H, m), 1.95 (1H, m), 2.50 (1H, m, partially obscured by DMSO-d5 signal), 2.95 (2H, m), 3.32 (2H, m), 3.73 (2H, m), 4.28 (3H, m), 7.26 (1H, d), 7.34 (1H, d), 7.42 (1H, d), 7.51 (2H, d), 7.60 (1H, d), 7.66 (1H, s), 7.73 (2H, d) 8.05 (1H, d), 9.26 (2H, br. s).

### Example 11

### 5′-Chloro-N-[(3S)-2-oxo-1-(2,3,4,5-tetrahydro-1H-2-benzazepin-8-yl)-3-pyrrolidinyl]-2,2′-bithiophene-5-sulfonamide hydrochloride

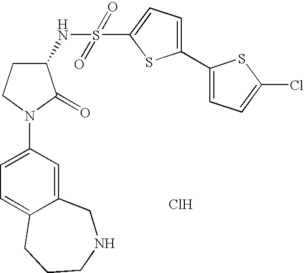

Prepared from 1,1-dimethylethyl 8-[(3S)-3-amino-2-oxo-1-pyrrolidinyl]-1,3,4,5-tetrahydro-2H-2-benzazepine-2-carboxylate (see Intermediate 17) and 5′-chloro-2,2′-bithiophene-5-sulfonyl chloride using a procedure similar to that described for Intermediate 9 followed by a deprotection procedure similar to that described for Example 8.

Mass spectrum: Found: MH⁺ 508, 510

H.p.l.c. Rₜ=2.74 min.

¹H NMR (d₆-DMSO) δ: 1.83 (3H, m), 2.32 (1H, m), 2.94 (2H, m), 3.33 (2H, m, partially obscured by HOD signal), 3.70 (2H, m), 4.28 (2H, s), 4.36 (1H, t), 7.21 (1H, m), 7.26 (1H, d), 7.38 (2H, m), 7.57 (1H, d), 7.64 (2H, m), 8.69 (1H, br.s), 9.14 (2H, br.s).

### Example 12

### 5-Chloro-N-[(3S)-2-oxo-1-(2,3,4,5-tetrahydro-1H-2-benzazepin-8-yl)-3-pyrrolidinyl]-1-benzothiophene-2-sulfonamide hydrochloride

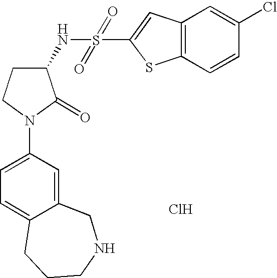

Prepared from 1,1-dimethylethyl 8-[(3S)-3-amino-2-oxo-1-pyrrolidinyl]-1,3,4,5-tetrahydro-2H-2-benzazepine-2-carboxylate (see Intermediate 17) and 5-chloro-1-benzothiophene-2-sulfonyl chloride using a procedure similar to that described for Intermediate 9 followed by a deprotection procedure similar to that described for Example 8.

Mass spectrum: Found: MH⁺ 476, 478

H.p.l.c. Rₜ=2.59 min.

¹H NMR (d₆-DMSO) δ: 1.84 (3H, m), 2.30 (1H, m), 2.93 (2H, m), 3.30 (2H, m), 3.69 (2H, m), 4.27 (2H, s), 4.40 (1H, t), 7.24 (1H, d), 7.55 (2H, d), 7.63 (1H, s), 8.04 (1H, d), 8.07 (1H, s), 8.30 (1H, s), 8.86 (1H, br.s), 9.21 (2H, br.s).

### Example 13

### (E)-2-(5-Chloro-2-thienyl)-N-methyl-N-[(3S)-2-oxo-1-(2,3,4,5-tetrahydro-1H-2-benzazepin-7-yl)-3-pyrrolidinyl]ethenesulfonamide hydrochloride

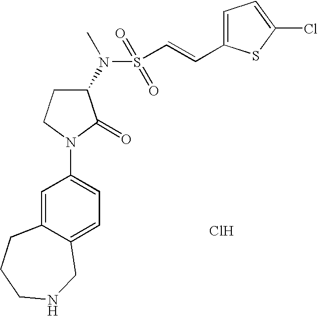

1,1-Dimethylethyl 7-{(3S)-3-[{[(E)-2-(5-chloro-2-thienyl)ethenyl]sulfonyl}(methyl)amino]-2-oxo-1-pyrrolidinyl}-1,3,4,5-tetrahydro-2H-2-benzazepine-2-carboxylate (see Intermediate 19) (88 mg, 0.15 mmol) was dissolved in 2M hydrogen chloride in diethyl ether (4 ml) under nitrogen and the mixture was left at room temperature for 18.75 h. The mixture was filtered and the solid was washed with diethyl ether and dried in vacuo at room temperature to give the title compound as a white solid (67 mg).

Mass spectrum: Found [MH⁺]⁻ 466, 468

H.p.l.c. Rₜ 2.55 min

¹H NMR (DMSO-d6) δ: 1.80-1.90 (2H, m); 2.12-2.24 (1H, m); 2.32-2.41 (1H, m); 2.74 (3H, s); 2.94-3.00 (2H, m); 3.25-3.31 (2H, m, partially obscured by HOD signal); 3.72-3.84 (2H, m); 4.29 (2H, br. s); 4.86 (1H, dd); 6.96 (1H, d); 7.22 (1H, d); 7.40-7.59 (5H, m); 9.00 (2H, br.s)

### Example 14 and Example 15

### Ethyl N-{[(E)-2-(5-chloro-2-thienyl)ethenyl]sulfonyl}-N-[(3S)-2-oxo-1-(2,3,4,5-tetrahydro-1H-2-benzazepin-7-yl)-3-pyrrolidinyl]glycinate and 1,1,Dimethylethyl N-{[(E)-2-(5-chloro-2-thienyl)ethenyl]sulfonyl}-N-[(3S)-2-oxo-1-(2,3,4,5-tetrahydro-1H-2-benzazepin-7-yl)-3-pyrrolidinyl]glycinate

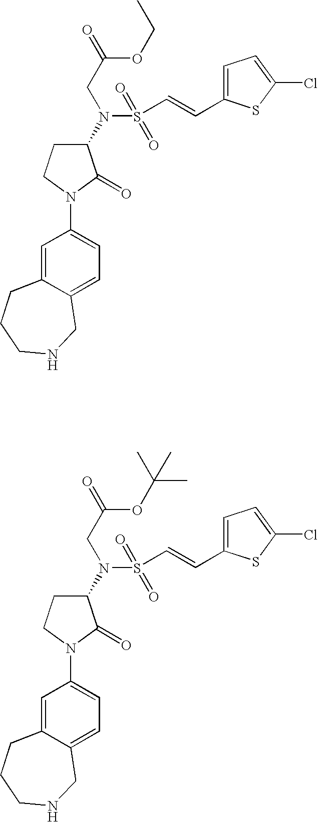

1,1-Dimethylethyl 7-[(3S)-3-({[(E)-2-(5-chloro-2-thienyl)ethenyl]sulfonyl}{2-[(1,1-dimethylethyl)oxy]-2-oxoethyl}amino)-2-oxo-1-pyrrolidinyl]-1,3,4,5-tetrahydro-2H-2-benzazepine-2-carboxylate (see Intermediate 20) (100 mg, 0.15 mmol) was dissolved in 2M hydrogen chloride in diethyl ether (2 ml). After 5 min, anhydrous EtOH (5 ml) was added. Further 2M hydrogen chloride in diethyl ether (3 ml) was added after 17.25 h, followed by an additional 2 ml after a further 6.5 h and the mixture was left at room temperature for 18 h after the addition of the third portion of 2M hydrogen chloride in diethyl ether. The solvents were evaporated under reduced pressure, collected by filtration washed with EtOH and dried in vacuo. The residue was purified by mass-directed autoprep to give the title compound (ethyl ester) as a pale yellow solid (40 mg)

¹H NMR (DMSO-d6) δ: 1.19 (3H, t); 1.60-1.78 (2H, m); 2.12-2.28 (1H, m); 2.35-2.51 (1H, m, partially obscured by DMSO-d5 signal); 2.86-2.94 (2H, m); 3.12-3.18 (2H, m); 3.64-4.15 (8H, m); 4.74-4.82 (1H, m); 6.98 (1H, d); 7.19-7.26 (2H, m); 7.36-7.57 (4H, m)

Mass spectrum: Found [MH⁺]⁻ 538, 540

H.p.l.c. Rₜ 2.68 min

and the title compound (1,1-dimethylethyl ester) as a pale cream solid (5 mg)

¹H NMR (MeOH-d4) δ: 1.46 (9H, s); 1.99-2.03 (2H, m); 2.25-2.37 (1H, m); 2.54-2.62 (1H, m); 3.04-3.10 (2H, m); 3.47 (2H, t); 3.76-4.02 (4H, m); 4.30-4.42 (2H, m); 4.80 (1H, dd); 6.88 (1H, d); 7.01 (1H, d); 7.25 (1H, d); 7.39 (1H, d); 7.49-7.56 (2H, m); 7.53 (1H, d)

Mass spectrum: Found [MH⁺]⁻ 566, 568

H.p.l.c. Rₜ 2.84 min

### Example 16

### N-{[(E)-2-(5-Chloro-2-thienyl)ethenyl]sulfonyl}-N-[(3S)-2-oxo-1-(2,3,4,5-tetrahydro-1H-2-benzazepin-7-yl)-3-pyrrolidinyl]glycine hydrochloride

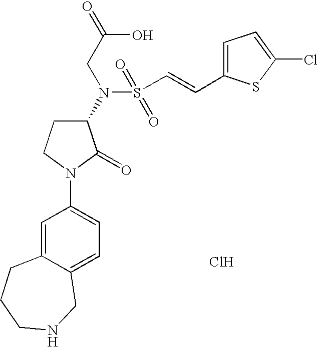

1,1-Dimethylethyl 7-[(3S)-3-({[(E)-2-(5-chloro-2-thienyl)ethenyl]sulfonyl}{2-[(1,1-dimethylethyl)oxy]-2-oxoethyl}amino)-2-oxo-1-pyrrolidinyl]-1,3,4,5-tetrahydro-2H-2-benzazepine-2-carboxylate (see Intermediate 20) (102 mg, 0.15 mmol) was dissolved in 2M hydrogen chloride in diethyl ether (4 ml) under nitrogen at room temperature. The mixture was left to stand at room temperature overnight in a flask covered with foil. After 18.25 h the mixture was filtered and the solid was washed with diethyl ether, dried in vacuo then treated with further 2M hydrogen chloride in diethyl ether (2 ml) and allowed to stand at room temperature for a further 16 h. The mixture was filtered, and the solid was washed with diethyl ether and dried in vacuo at room temperature to give the title compound as a pale cream solid (58 mg)

¹H NMR (DMSO-d6) δ: 1.79-1.90 (2H, m); 2.14-2.26 (1H, m); 2.36-2.46 (1H, m,); 2.93-3.01 (2H, m); 3.36-3.42 (2H, m); 3.66-4.00 (4H, m); 4.28 (2H, br. s); 4.79 (1H, dd); 6.96 (1H, d); 7.21 (1H, d); 7.39-7.58 (5H, m); 9.03 (2H, br. s); 12.79 (1H, br. s)

Mass spectrum: Found [MH⁺]⁻ 510, 512

H.p.l.c. Rₜ 2.43 min

### Example 17

### (E)-2-(5-Chloro-2-thienyl)-N-(2-hydroxyethyl)-N-[(3S)-2-oxo-1-(2,3,4,5-tetrahydro-1H-2-benzazepin-7-yl)-3-pyrrolidinyl]ethenesulfonamide hydrochloride

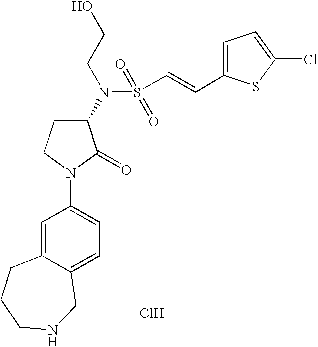

1,1-Dimethylethyl 7-{(3S)-3-[{[(E)-2-(5-chloro-2-thienyl)ethenyl]sulfonyl}(2-hydroxyethyl)amino]-2-oxo-1-pyrrolidinyl}-1,3,4,5-tetrahydro-2H-2-benzazepine-2-carboxylate (see Intermediate 21) (27 mg) was dissolved in 4M hydrogen chloride in 1,4-dioxane (2 ml) and the solution was left at room temperature overnight (protected from light). The solvent and excess of hydrogen chloride were blown off using a stream of nitrogen to give the title compound (16 mg).

¹H NMR (DMSO-d6) δ: 1.95-2.05 (2H, m); 2.39-2.60 (2H, m); 3.03-3.11 (2H, m); 3.46-3.51 (2H, m); 3.62-3.77 (4H, m); 3.80-3.99 (2H, m); 4.19-4.22 (1H, m); 4.36 (2H, br. s); 4.79 (1H, t); 6.88 (1H, d); 7.01 (1H, d); 7.24 (1H, d); 7.40 (1H, d); 7.41-7.55 (2H, m); 7.63 (1H, br. s)

Mass spectrum: Found [MH⁺]⁻ 496, 498

H.p.l.c. Rₜ 2.44 min

### Example 18

### 1-Methylethyl N-{[(E)-2-(5-chloro-2-thienyl)ethenyl]sulfonyl}-N-[(3S)-2-oxo-1-(2,3,4,5-tetrahydro-1H-2-benzazepin-7-yl)-3-pyrrolidinyl]glycinate hydrochloride

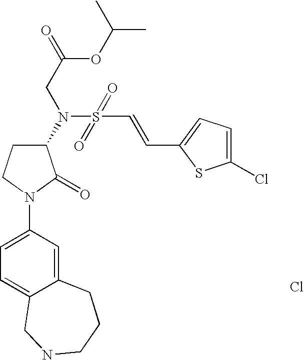

1,1-Dimethylethyl 7-[(3S)-3-({[(E)-2-(5-chloro-2-thienyl)ethenyl]sulfonyl}{2-[(1-methylethyl)oxy]-2-oxoethyl}amino)-2-oxo-1-pyrrolidinyl]-1,3,4,5-tetrahydro-2H-2-benzazepine-2-carboxylate (0.80 g, 1.22 mmol) (see Intermediate 22) was dissolved in isopropanol (10 ml) and 2M hydrogen chloride in diethyl ether (50 ml) was stirred under nitrogen in the dark at room temperature overnight. Solvent was removed in vacuo and the residual solid was triturated with diethyl ether (3×10 ml), then collected by filtration dried in vacuo at 45° C. to give the title compound as a white solid (0.607 g).

¹H NMR (DMSO-d6) δ: 1.19 (6H, m); 1.85 (2H, m); 2.20 (1H, m); 2.42 (1H, m, partially obscured by DMSO-d5 signal); 2.97 (2H, m); 3.28-3.39 (2H, m, obscured by HOD signal), 3.68-3.80 (2H, m); 3.89 and 4.01 (2H, ABq); 4.27 (2H, br.s); 4.80 (1H, t), 4.92 (1H, m), 6.98 (1H, d); 7.21 (1H, d), 7.39-7.56 (5H, m); 9.16 (2H, br.s)

Mass spectrum: Found [MH⁺] 552

H.p.l.c. Rₜ 2.78 min

### Example 19

### Ethyl N-{[(E)-2-(5-chloro-2-thienyl)ethenyl]sulfonyl}-N-[(3S)-2-oxo-1-(2,3,4,5-tetrahydro-1H-2-benzazepin-7-yl)-3-pyrrolidinyl]alaninate hydrochloride (isomer 1)

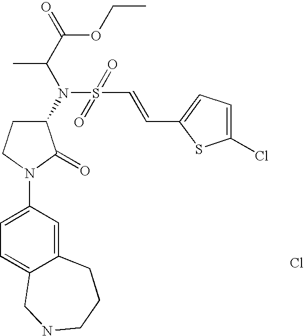

1,1-Dimethylethyl 7-((3S)-3-{{[(E)-2-(5-chloro-2-thienyl)ethenyl]sulfonyl}[2-(ethyloxy)-1-methyl-2-oxoethyl]amino}-2-oxo-1-pyrrolidinyl)-1,3,4,5-tetrahydro-2H-2-benzazepine-2-carboxylate (isomer 1) (0.03 g, 0.046 mmol) (see Intermediate 23) was dissolved in ethanol (1 ml) and 2M hydrogen chloride in diethyl ether (5 ml) was added under nitrogen and the mixture was stirred in the dark at room temperature overnight. Solvent was removed in vacuo and the residual solid was collected by filtration and dried in vacuo at 45° C. to give the title compound (0.023 g) as a white solid.

¹H NMR (DMSO-d6) δ: 1.13 (3H, t); 1.42 (3H, d), 1.86 (2H, br.m); 2.25 (1H, m); 2.50 (1H, obscured by DMSO-d5 signal); 2.98 (2H, m); 3.32 (2H, obscured by HOD signal), 3.77 (2H, m); 4.04 (2H, q); 4.29 (2H, s); 4.42 (1H, q), 4.55 (1H, t), 7.01 (1H, d); 7.22 (1H, d), 7.41-7.58 (5H, m); 9.01 (2H, br.s)

Mass spectrum: Found [MH⁺] 552

H.p.l.c. Rₜ 2.75 min

### Example 20

### Ethyl N-{[(E)-2-(5-chloro-2-thienyl)ethenyl]sulfonyl}-N-[(3S)-2-oxo-1-(2,3,4,5-tetrahydro-1H-2-benzazepin-7-yl)-3-pyrrolidinyl]alaninate hydrochloride (isomer 2)

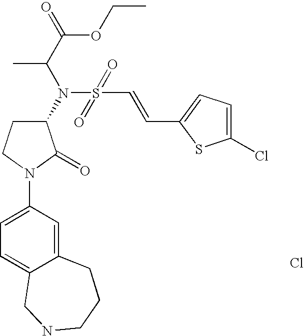

1,1-Dimethylethyl 7-((3S)-3-{{[(E)-2-(5-chloro-2-thienyl)ethenyl]sulfonyl}[2-(ethyloxy)-1-methyl-2-oxoethyl]amino}-2-oxo-1-pyrrolidinyl)-1,3,4,5-tetrahydro-2H-2-benzazepine-2-carboxylate (isomer 2) (0.05 g, 0.076 mmol) (see Intermediate 24) was dissolved in ethanol (1 ml) and 2M hydrogen chloride in diethyl ether (5 ml) was added under nitrogen and the mixture was stirred in the dark at room temperature overnight. Solvent was removed in vacuo and the residual solid was transferred as an ether suspension to a bottle then blown dry with nitrogen and dried in vacuo at 45° C. overnight to give the title compound (0.033 g) as a white solid.

¹H NMR (DMSO-d6) δ: 1.2 (3H, t); 1.49 (3H, d), 1.86 (2H, br.s); 2.42-2.55 (2H, obscured by DMSO-d5 signal); 2.97 (2H, m); 3.31 (2H, obscured by HOD signal), 3.75 (2H, m); 4.12 (2H, q); 4.28 (2H, s); 4.37 (1H, q), 4.58 (1H, t), 6.91 (1H, d); 7.22 (1H, d), 7.40-7.50 (3H, m), 7.56 (1H, d), 7.62 (1H, s), 9.05 (2H, br.s).

Mass spectrum: Found [MH⁺] 552

H.p.l.c. Rₜ 2.75 min

### Example 21

### (E)-2-(5-Chloro-2-thienyl)-N-[(3S)-1-(6-fluoro-2,3,4,5-tetrahydro-1H-2-benzazepin-7-yl)-2-oxo-3-pyrrolidinyl]ethenesulfonamide hydrochloride

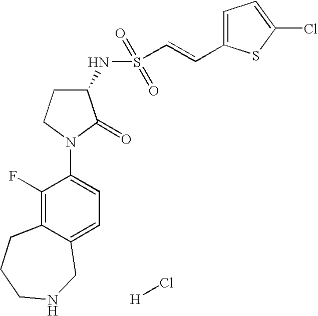

Prepared from 1,1-Dimethylethyl 7-[(3S)-3-amino-2-oxo-1-pyrrolidinyl]-6-fluoro-1,3,4,5-tetrahydro-2H-2-benzazepine-2-carboxylate (40 mg) (see Intermediate 37) and (E)-2-(5-chloro-2-thienyl)ethanesulfonyl chloride using a procedure similar to that described for Intermediate 9 followed by a deprotection procedure similar to that described for Example 1.

Mass spectrum: Found: MH⁺ 470, 472

H.p.l.c. Rₜ=2.44 min.

¹H nmr (d₃-MeOD) δ: 1.99 (2H, brm), 2.16 (1H, m), 2.65 (1H, m), 3.13 (2H, m), 3.50 (2H, m), 3.81 (2H, m), 4.35 (1H, m), 4.43 (2H, s), 6.88 (1H, d), 6.99 (1H, d), 7.21 (1H, d) 7.28 (2H, m), 7.51 (1H, d).

### Example 22

### 6-Chloro-N-[(3S)-1-(6-fluoro-2,3,4,5-tetrahydro-1H-2-benzazepin-7-yl)-2-oxo-3-pyrrolidinyl]-2-naphthalenesulfonamide hydrochloride

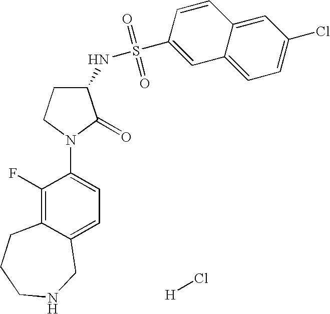

Prepared from 1,1-Dimethylethyl 7-[(3S)-3-amino-2-oxo-1-pyrrolidinyl]-6-fluoro-1,3,4,5-tetrahydro-2H-2-benzazepine-2-carboxylate (38.0 mg) (see Intermediate 37) and 6-chloro-2-naphthalenesulfonyl chloride using a procedure similar to that described for Intermediate 9 followed by a deprotection procedure similar to that described for Example 21.

Mass spectrum: Found: MH⁺ 488, 490

H.p.l.c. Rₜ=2.60 min

¹H nmr (d₃-MeOD) δ: 1.96 (3H, m), 2.40 (1H, m), 3.09 (2H, m), 3.47 2H, m), 3.60 (2H, m), 4.36 (1H, m), 4.39 (2H, s), 7.22 (2H, m), 7.59 (1H, dd), 7.88-8.13 (4H, m), 8.53 (1H, s).

### In Vitro Assay for Inhibition of Factor Xa

Compounds of the present invention were tested for their Factor Xa inhibitory activity as determined in vitro by their ability to inhibit human Factor Xa in a fluorogenic assay, using Rhodamine 110, bis-CBZ-glycylglycyl-L-arginine amide as the fluorogenic substrate. Compounds were diluted from a 10 mM stock solution in dimethylsulfoxide at appropriate concentrations. Assay was performed at room temperature using buffer consisting of: 50 mM Tris-HCl, 150 mM NaCl, 5 mM CaCl₂, pH 7.4 containing human Factor Xa (final concentration of 0.0003 U.ml-1). Compound and enzyme were preincubated for 15 min prior to addition of the substrate (final concentration of 10 μM). The reaction was stopped after 3 hrs with the addition of H-D-Phe-Pro-Arg-Chloromethylketone. An LJL-Analyst fluorimeter was used to monitor fluorescence with 485 nm excitation/535 nm emission. To obtain IC₅₀ values the data were analysed using ActivityBase® and XLfit®.

### Calculation of Ki Values:

Ki=IC₅₀/(1+[Substrate]/Km)

The Ki value for the above assay can be obtained by dividing the IC₅₀ value by 1.6.

All of the synthetic Example compounds tested by the above described in vitro assay were found to exhibit Factor Xa inhibitory activity (Examples 1-22). Preferably, compounds have a Ki value of less than 1 μM (Examples 1-22). More preferably, compounds have a Ki value of less than 0.1 μM (Examples 1-22). Most preferably, compounds have a Ki value of less than 50 nM (Examples 1-22).

Method for measurement of Prothrombin Time (PT)

Blood was collected into a sodium citrate solution (ratio 9:1) to give a final concentration of 0.38% citrate. Plasma was generated by centrifugation of citrated blood samples at 1200×g for 20 min at 4° C. and stored at −20° C. until use. PT analysis was conducted using plasma pooled from 4 separate donors (2 male and 2 female).

The PT test was performed using the BCS Coagulation Analyzer (Dade Behring). For assay, 50 ul of plasma containing test compound at concentrations ranging from 0.03 to 100 uM (made from a 100 uM stock containing 1% DMSO in plasma) was combined with 100 ul of Thromboplastin C Plus (Dade Behring). Upon addition of the reagents, absorbance at 405 nm was monitored and time to clot formation is determined (normal range for human plasma is 10.6-12.4 seconds).

All of the synthetic Example compounds tested by the above described assay were found to exhibit activity (Examples 1-22). Preferably, compounds have a 1.5×PT less than 60 μl.

### Human Prothrombinase Assay

Example 1 compound was tested in a prothrombinase assay based on the human prothrombinase complex (includes the cofactor factor Va, phospholipid vesicles and prothrombin) and found to exhibit activity with a Ki value less than 20 nM.

The prothrombinase assay was assembled by adding phosphatidyl serine/phosphatidyl choline vesicles (1:1 stoichiometry −50 μM total), CaCl₂ (5 mM), FXa (0.01 nM), FVa (5 nM) into a Tris/NaCl buffer (0.05M, pH 7.4, NaCl 0.15M). This mixture was incubated on ice for 20 minutes. Example 1 was then added to the mixture over a concentration range of 0.0001-10 μM in a 96 well plate and incubated for 5 minutes at room temperature. The reaction was initiated by the addition of prothrombin (1.2 μM) and incubated for 2 minutes at room temperature. The reaction was then terminated with a Tris/EDTA buffer (0.05M, pH 7.4, EDTA 0.083M). An aliquot of terminated reaction mixture was then transferred to a second 96 well plate containing the thrombin chromogenic substrate S-2238 (110 μM), and the release of para-nitroanilide as a measure of thrombin activity was determined in a spectrophotometer microplate reader at a wavelength of 405 nm over 5 minutes. Data were fit to a single-site competition model, and an inhibition constant calculated using the Cheng-Prusoff equation (Cheng Y., Prusoff W. H., Biochem. Pharmacol. 22: 3099-3108, 1973).

### General Purification and Analytical Methods

### LC/MS Method

Analytical HPLC was conducted on a Supelcosil LCABZ+PLUS column (3 μm, 3.3 cm×4.6 mm ID) eluting with 0.1% HCO₂H and 0.01M ammonium acetate in water (solvent A), and 95% MeCN and 0.05% HCO₂H in water (solvent B), using the following elution gradient 0-0.7 min 0% B, 0.7-4.2 min 0→100% B, 4.2-5.3 min 100% B, 5.3-5.5 min 100→0% B at a flow rate of 3 ml/min (System 1). The mass spectra (MS) were recorded on a Fisons VG Platform mass spectrometer using electrospray positive ionisation [(ES+ve to give MH⁺ and M(NH₄)⁺ molecular ions] or electrospray negative ionisation [(ES−ve to give (M−H)⁻ molecular ion] modes.

¹H NMR spectra were recorded using a Bruker DPX 400 MHz spectrometer using tetramethylsilane as the external standard.

Hydrophobic frits refers to filtration tubes sold by Whatman.

SPE (solid phase extraction) refers to the use of cartridges sold by International Sorbent Technology Ltd. Silica SPE and SCX SPE were used.

Combi Flash® Companion® refers to an automated purification system sold by ISCO Inc.

Redisep® silica columns refer to pre-packed columns sold by ISCO Inc.

Biotage™ chromatography refers to purification carried out using equipment sold by Dyax Corporation (either the Flash 40i or Flash 150i) and cartridges pre-packed with KPSil™.

1. A compound of formula (I): 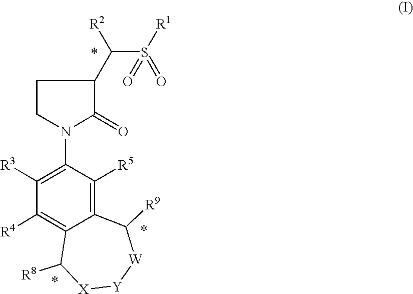
wherein:
R¹ represents a group selected from: 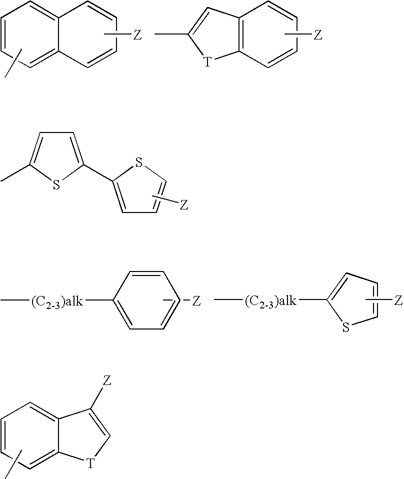
each ring of which optionally contains a further heteroatom N,
Z is an optional substituent halogen,
alk is alkylene or alkenylene, and
T represents S, O or NH;
R² is hydrogen, —C₁₋₆alkyl, —C₁₋₃alkylCONR^{a}R^{b}, —C₁₋₃alkylCO₂C₁₋₄alkyl, —CO₂C₁₋₄alkyl, —C₁₋₃alkylOH, —C₁₋₃alkylOC₁₋₃alkyl, or —C₁₋₃alkylCO₂H;
R^{a} and R^{b} independently are hydrogen, —C₁₋₆alkyl, or together with the N atom to which they are bonded form a 5-, 6- or 7-membered non-aromatic heterocyclic ring optionally containing an additional heteroatom selected from O, N and S, optionally substituted by C₁₋₄ alkyl, and optionally the S heteroatom is substituted by S(O)ₙ;
n is 0-2;
one of W, X and Y is —N(R⁶)— and the others are —CH(R⁷)—.
R³, R⁴ and R⁵ independently are hydrogen, C₁₋₄alkyl or halogen;
R⁶ is hydrogen or C₁₋₄alkyl;
R⁷, R⁸ and R⁹ independently are hydrogen or C₁₋₄alkyl;
and pharmaceutically acceptable derivative(s) thereof.

2. A compound according to claim 1 and pharmaceutically acceptable derivative(s) thereof wherein:
R¹ is a group selected from: 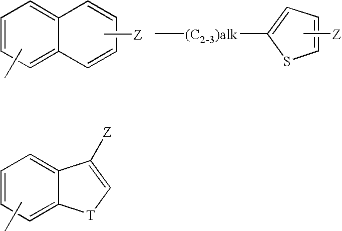
each ring of which optionally contains a further heteroatom N,
Z represents an optional substituent halogen,
alk represents alkylene or alkenylene, and
T represents S, O or NH.

3. A compound according to claim 1 and pharmaceutically acceptable derivative(s) thereof, wherein one of W, X and Y is —NH— and the others are —CH₂—.

4. A compound according to claim 1 and pharmaceutically acceptable derivative(s) thereof, wherein R² is hydrogen, —C₁₋₆alkyl, —C₁₋₃alkylCO₂C₁₋₄alkyl, or —C₁₋₃alkylCO₂H.

5. A compound according to claim 4 and pharmaceutically acceptable derivative(s) thereof, wherein R² is —C₁₋₃ alkylCO₂C₁₋₄alkyl or —C₁₋₃alkylCO₂H.

6. A compound according to claim 4 and pharmaceutically acceptable derivative(s) thereof, wherein wherein R² is hydrogen.

7. A compound according to claim 1 selected from:
(E)-2-(5-Chloro-2-thienyl)-N-[(3S)-2-oxo-1-(2,3,4,5-tetrahydro-1H-2-benzazepin-7-yl)-3-pyrrolidinyl]ethenesulfonamide;
6-Chloro-N-[(3S)-2-oxo-1-(2,3,4,5-tetrahydro-1H-2-benzazepin-7-yl)-3-pyrrolidinyl]-2-naphthalenesulfonamide;
3-Chloro-N-[(3S)-2-oxo-1-(2,3,4,5-tetrahydro-1H-2-benzazepin-7-yl)-3-pyrrolidinyl]-1H-indole-6-sulfonamide;
6-Chloro-N-[(3S)-1-(2-methyl-2,3,4,5-tetrahydro-1H-2-benzazepin-7-yl)-2-oxo-3-pyrrolidinyl]-2-naphthalenesulfonamide;
(E)-2-(5-Chloro-2-thienyl)-N-[(3S)-2-oxo-1-(2,3,4,5-tetrahydro-1H-3-benzazepin-7-yl)-3-pyrrolidinyl]ethenesulfonamide;
6-Chloro-N-[(3S)-2-oxo-1-(2,3,4,5-tetrahydro-1H-3-benzazepin-7-yl)-3-pyrrolidinyl]-2-naphthalenesulfonamide;
3-Chloro-N-[(3S)-2-oxo-1-(2,3,4,5-tetrahydro-1H-3-benzazepin-7-yl)-3-pyrrolidinyl]-1H-indole-6-sulfonamide;
(E)-2-(5-Chloro-2-thienyl)-N-[(3S)-2-oxo-1-(2,3,4,5-tetrahydro-1H-2-benzazepin-8-yl)-3-pyrrolidinyl]ethenesulfonamide;
6-Chloro-N-[(3S)-2-oxo-1-(2,3,4,5-tetrahydro-1H-2-benzazepin-8-yl)-3-pyrrolidinyl]-2-naphthalenesulfonamide;
(E)-2-(4-Chlorophenyl)-N-[(3S)-2-oxo-1-(2,3,4,5-tetrahydro-1H-2-benzazepin-8-yl)-3-pyrrolidinyl]ethenesulfonamide;
5′-Chloro-N-[(3S)-2-oxo-1-(2,3,4,5-tetrahydro-1H-2-benzazepin-8-yl)-3-pyrrolidinyl]-2,2′-bithiophene-5-sulfonamide;
5-Chloro-N-[(3S)-2-oxo-1-(2,3,4,5-tetrahydro-1H-2-benzazepin-8-yl)-3-pyrrolidinyl]-1-benzothiophene-2-sulfonamide;
(E)-2-(5-Chloro-2-thienyl)-N-methyl-N-[(3S)-2-oxo-1-(2,3,4,5-tetrahydro-1H-2-benzazepin-7-yl)-3-pyrrolidinyl]ethenesulfonamide;
Ethyl N-{[(E)-2-(5-chloro-2-thienyl)ethenyl]sulfonyl}-N-[(3S)-2-oxo-1-(2,3,4,5-tetrahydro-1H-2-benzazepin-7-yl)-3-pyrrolidinyl]glycinate;
1,1-Dimethylethyl N-{[(E)-2-(5-chloro-2-thienyl)ethenyl]sulfonyl}-N-[(3S)-2-oxo-1-(2,3,4,5-tetrahydro-1H-2-benzazepin-7-yl)-3-pyrrolidinyl]glycinate;
N-{[(E)-2-(5-Chloro-2-thienyl)ethenyl]sulfonyl}-N-[(3S)-2-oxo-1-(2,3,4,5-tetrahydro-1H-2-benzazepin-7-yl)-3-pyrrolidinyl]glycine;
(E)-2-(5-Chloro-2-thienyl)-N-(2-hydroxyethyl)-N-[(3S)-2-oxo-1-(2,3,4,5-tetrahydro-1H-2-benzazepin-7-yl)-3-pyrrolidinyl]ethenesulfonamide;
1-Methylethyl N-{[(E)-2-(5-chloro-2-thienyl)ethenyl]sulfonyl}-N-[(3S)-2-oxo-1-(2,3,4,5-tetrahydro-1H-2-benzazepin-7-yl)-3-pyrrolidinyl]glycinate;
Ethyl N-{[(E)-2-(5-chloro-2-thienyl)ethenyl]sulfonyl}-N-[(3S)-2-oxo-1-(2,3,4,5-tetrahydro-1H-2-benzazepin-7-yl)-3-pyrrolidinyl]alaninate;
(E)-2-(5-Chloro-2-thienyl)-N-[(3S)-1-(6-fluoro-2,3,4,5-tetrahydro-1H-2-benzazepin-7-yl)-2-oxo-3-pyrrolidinyl]ethenesulfonamide;
6-Chloro-N-[(3S)-1-(6-fluoro-2,3,4,5-tetrahydro-1H-2-benzazepin-7-yl)-2-oxo-3-pyrrolidinyl]-2-naphthalenesulfonamide;
and pharmaceutically acceptable derivative(s) thereof.

8. A compound according to claim 7, selected from (E)-2-(5-Chloro-2-thienyl)-N-[(3S)-2-oxo-1-(2,3,4,5-tetrahydro-1H-2-benzazepin-7-yl)-3-pyrrolidinyl]ethenesulfonamide and pharmaceutically acceptable derivative(s) thereof.

9. A compound to claim 7, which is (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hemi-succinate.

10. A compound according to claim 7, which is (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride.

11. A compound according to claim 7, which is (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide hydrochloride hydrate.

12. A compound according to claim 7, which is (E)-2-(5-chloro-2-thienyl)-N-[(3S)-2-oxo-1-(2,3,4,5-tetrahydro-1H-2-benzazepin-7-yl)-3-pyrrolidinyl]ethenesulfonamide.

13. A pharamaceutical composition comprising a compound according to claim 1 together with at least one pharmaceutical carrier and/or excipient.

14. A method of treating a patient suffering from a condition susceptible to amelioration by a Factor Xa inhibitor comprising administering to the patient a therapeutically effective amount of a compound according to claim 1.

15. A method of treating a patient suffering from acute coronary syndromes, pulmonary embolism, deep vein thrombosis and the thromboembolic events associated with atrial fibrillation comprising adminstering to the patient a therapeutically effective amount of a compound according to claim 1.

16. A process for preparing a compound according to claim 1, comprising reacting a compound of fourmula (II) or an acid addition salt thereof with a compound of formula (III): 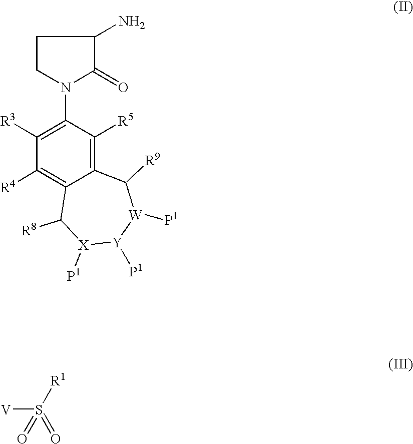
wherein V is a suitable leaving group.
